(19) United States
(12) Patent Application Publication
(10) Pub. No.: US 2025/0299818 A1
(43) Pub. Date: Sep. 25, 2025

(54) DEPLOYMENT AND USE OF A CONTINUOUS ANALYTE MONITORING SYSTEM FOR IMPROVED HOME MONITORING AND READMISSION OPTIMIZATION

(71) Applicant: Abbott Diabetes Care Inc., Alameda, CA (US)

(72) Inventors: Anup J. SHAH, Portland, OR (US); Matthew BATES, New Hope, PA (US); Paul R. E. JARVIS, Pudsey (GB); Naveen Venkata THURAMALLA, Dublin, CA (US)

(21) Appl. No.: 19/066,786

(22) Filed: Feb. 28, 2025

Related U.S. Application Data
(60) Provisional application No. 63/569,606, filed on Mar. 25, 2024.

(51) Int. Cl. G16H 50/20; A61B 5/00; A61B 5/145; G16H 10/60; G16H 40/20; G16H 40/60; G16H 50/50

(52) U.S. Cl. CPC G16H 50/20; A61B 5/14546; A61B 5/7275; A61B 5/746; G16H 10/60; G16H 40/20; G16H 40/60; G16H 50/50; A61B 5/14532

(57) ABSTRACT

Disclosed herein are system, method, and computer program product embodiments to an improved alert and recommendation system for reducing patient readmission via the detection and treatment of patient conditions based on continuous analyte data. The disclosed techniques utilize analyte data, such as lactate, glucose, and creatinine, provided from a continuous analyte sensor to predict patient outcomes and generate recommendations for reducing patient readmission in a hospital and home setting. The disclosed system allows for early and non-invasive prediction of patient outcomes and the subsequent generation of recommended actions to facilitate patient intervention with the goal of reducing readmission of the patient.

### CROSS-REFERENCE TO RELATED APPLICATION

This application claims priority to U.S. Provisional Application 63/569,606, filed on Mar. 25, 2024, which is incorporated by reference in its entirety.

### FIELD

This disclosure generally relates to continuous analyte sensors, including in vivo analyte sensors for sensing and monitoring analytes in a bodily fluid, and the use of such sensors for predicting patient outcomes and improving alerts and notifications through updated visualizations based on the predicted patient outcomes.

### BACKGROUND

Current methodologies for monitoring patients managing chronic or acute illnesses can present challenges because many monitoring techniques rely on invasive procedures, such as serial blood draws or quasi-surgical or surgical interventions. More problematically, and because this type of monitoring is often reactive in nature, and can be spaced apart by hours or days, important early warning signs which might otherwise prompt urgent physician intervention can be missed either by the patient who may, subjectively, “feel fine,” or by the treating physician who cannot observe visual or other changes that would indicate an impending or more immediate severe disease state. Thus, reactive, infrequent monitoring can leave large gaps for which no data is available, leaving a physician without critical information about the patient's condition.

### SUMMARY

The present disclosure provides a decision support system that leverages continuous analyte monitoring techniques for continuously monitoring a patient's analyte levels in order to generate predicted patient outcomes based on the continuously monitored data, and facilitate earlier treatment decisions for the patient based on the predicted patient outcome.

The decision support system can be deployed in various settings, including in both in-patient and out-patient settings. Specifically, as described herein, the system can implement a machine learning supported decision support model for generating outputs utilized in a hospital setting to provide real-time decisions support to treating physicians in order to provide real-time inpatient support. Alternatively, the decision support system described herein can be used at home in order to provide patients or physicians with decisions support in order to effectively reduce readmission events through real-time analyte monitoring. A readmission event is an event where a user is admitted as a patient into a hospital within a predetermined period of time after the user is discharged from the hospital (e.g. within 30 days of discharge). The decision support model is implemented in combination with a continuous analyte monitoring system and configured to provide alerts and notifications to designated devices based on detected medical conditions. Also disclosed herein are systems, methods, and computer program product embodiments providing an improved alert and recommendation system for reducing patient readmission via the detection and treatment of patient conditions based on continuous analyte data. The techniques described herein utilize analyte data, such as lactate, glucose, and creatinine, provided from a continuous analyte sensor to predict patient outcomes and generate recommendations for reducing patient readmission in a hospital and home setting. The disclosed system allows for early and non-invasive prediction of patient outcomes and the subsequent generation of recommended actions to facilitate patient intervention with the goal of reducing readmission of the patient.

Suitable continuous analyte sensors are described, variously, in U.S. Pat. Nos. 9,914,952, 10,392,647, 11,091,788, 12,076,145, 12,004,858, and U.S. Patent Publication No. 2021/0219885, all of which are incorporated by reference in their entirety.

Examples of such continuous analyte sensors include analyte sensors employing multiple enzymes for detection and in which multiple enzymes can function independently or in concert to detect one or more analytes. A number of advantages can be realized by incorporating multiple enzymes in an analyte sensor. In some sensor configurations suitable for the present disclosure, the multiple enzymes can facilitate independent detection of multiple analytes, such as glucose and lactate. Membranes configured to provide tailored permeability for multiple analytes, which can facilitate analyte detection with a single analyte sensor by levelizing the sensor's sensitivity toward each analyte. In other sensor configurations, multiple enzymes can be chosen to function in concert to facilitate detection of a single analyte of interest, which may otherwise be problematic or impossible to assay using a single enzyme. In any event, fewer electrodes may be needed to detect a given analyte or set of analytes than would otherwise be feasible.

Another example of a continuous analyte sensor includes an electrochemical analyte sensor utilizing a lactate-responsive enzyme for lactate detection and quantification. The electrochemical analyte sensor is adapted to be at least partially inserted into a tissue of interest, such as within the dermal or subcutaneous layer of the skin and can comprise a sensor tail of sufficient length for insertion to a desired depth in a given tissue. The sensor tail can comprise a working electrode and one or more active areas (sensing regions/spots or sensing layers) located upon the working electrode and that are active for sensing an analyte of interest, particularly lactate. According to one or more embodiments, each active area of the electrochemical analyte sensor may comprise a lactate-responsive enzyme, suitable examples of which may include lactate oxidase or lactate dehydrogenase. The active areas may include a polymeric material to which the enzyme is covalently bonded, according to some embodiments. In various embodiments, lactate may be monitored in any biological fluid of interest such as dermal fluid, interstitial fluid, plasma, blood, lymph, synovial fluid, cerebrospinal fluid, saliva, bronchoalveolar lavage, amniotic fluid, or the like. In particular embodiments, the analyte sensors of the present disclosure may be adapted for assaying dermal fluid or interstitial fluid.

Another example of a continuous analyte sensor includes analyte sensors employing multiple enzymes for detection of multiple analytes and, more specifically, analyte sensors employing multiple working electrodes for detecting multiple analytes, e.g., glucose, β-hydroxybutyrate, uric acid, ketone, creatinine, ethanol, and lactate. Multiple sensors may also be employed to analyze multiple analytes. In one embodiment, a sensor includes at least two working electrodes and counter/reference electrodes. In another embodiment, the analyte detection system may contain multiple sensors. The system may contain a primary sensor with at least one, optionally at least two, working electrodes, a counter electrode, and a reference electrode. The system may also contain a sub-sensor that contains at least one, optionally at least two, optionally at least three, optionally at least four working electrodes, and does not contain a counter or reference electrode. The sub-sensor is placed implanted into the user in close proximity to the primary sensor, such that the sub-sensor is able to share the counter and reference electrodes in the primary sensor. The sub-sensor may be contained in the same sensor housing as the primary sensor. Optionally the sub-sensor may be placed in a separate sensor housing that is in close proximity to the sensor housing of the primary sensor, such that the primary sensor and sub-sensor share the same counter and reference electrodes. In an alternative embodiment, multiple sub-sensors may share the counter and reference electrodes of the primary sensor.

The detection of various analytes within an individual can be vital for monitoring the condition of their health and well-being. Deviation from normal analyte levels can often be indicative of an underlying physiological condition, such as a metabolic condition or illness, or exposure to particular environmental factors or stimuli. Glucose levels, for example, can be particularly important to detect and monitor in diabetic individuals. Alternatively, or additionally, blood lactate concentration can be an important analyte to monitor to predict patient outcomes in the decision support systems provided herein.

In vivo lactate levels (i.e. lactate concentration) can vary in response to numerous environmental or physiological factors including, for example, eating, physiological stress, exercise, sepsis or septic shock, infection, hypoxia, heart failure, polytrauma, tissue hypoperfusion, and the like. In the case of chronic, ongoing conditions, such as heart failure, periodic laboratory measurements of lactate levels may be sufficient to determine whether these conditions are increasing or decreasing in severity, and/or if the patient is responding to treatment. Other lactate-altering conditions, such as sepsis or septic shock, may be episodic in nature, in which case lactate levels may fluctuate very rapidly and irregularly. In some embodiments, other use-cases in which continuous analyte information, including lactate, can be used for predicting patient outcomes include reducing readmission events for patients, which has applicability in a hospital setting (e.g., when the patients are still admitted in the hospital and being evaluated for discharge readiness) and home setting (e.g., when the patients have been discharged from the hospital), and disease and deterioration detection in conditions such as sepsis and heart failure. Conventional laboratory measurements, and in particular, their timing—typically several hours apart if measured at all—are ill suited to provide lactate levels in instances where frequent analyte measurements may be required. Namely, lactate levels may have changed several times between successive measurements, and an abnormal lactate level may go undetected in such instances, thereby leading to potentially missed and/or delayed diagnoses, or in cases of significant changes, substantially negative patient outcomes. Examples of changes to lactate levels include rises, falls, and any sequence of combinations of rises and falls. In the case of rapidly fluctuating lactate levels, it can be desirable to measure an individual's lactate levels continuously, such as through using an implanted in vivo lactate sensor, such as those referenced earlier herein. Even if a lactate spike is observed when measuring lactate levels with periodic laboratory measurements, there often is no possibility of taking proactive actions to alleviate or remediate a particular condition leading to the elevated lactate levels. This can have significant consequences for a user's health and well-being in some cases.

In some embodiments, the decision support system includes a prediction model which predicts patient outcomes for different settings-such as a hospital setting, a home setting, disease detection, and high-risk surgery monitoring-based on at least the continuous analyte data. In some embodiments, the analyte can be any combination of lactate and another analyte, such as glucose or creatinine. In some embodiments, the prediction model also bases the predicted patient outcomes on other medical information associated with the patient such as the patient's medical history, prior and current treatments, prior and current vital signs, trend information, and health care provider (HCP) notes and preferences. The decision support system can be further configured to provide alerts, notifications, and/or recommendations for treatment based on any combination of the predicted patient outcome, HCP preferences, and communication settings. Depending on the type of setting, such as a hospital, the alerts, notifications, and/or recommendations can be transmitted to different devices, such as patient monitoring devices (e.g., bedside monitors), HCP devices, and nurses devices. In some embodiments, the generated alerts, notifications, and recommendations can be used as part of the readmission reduction process.

Continuous lactate monitoring can also be advantageous in individuals with chronic, slowly changing lactate levels as well. For example, continuous lactate monitoring can avoid the pain and expense associated with conducting multiple blood draws for assaying lactate levels. Continuous lactate monitoring is additionally advantageous in that it is less resource intensive, in terms of staff and equipment usage, and more resource efficient than conventional methods. Continuous lactate monitoring is even further advantageous over conventional methods due to its ability to generate a more complete picture of the patients' disease state, allowing for better treatment option recommendation, and its ability to detect signs of patient deterioration earlier, which leads to lower patient mortality and increased speed of patient recovery.

According to some embodiments, a continuous analyte monitoring (CAM) system is configured with one or more analyte sensors in communication with one or more reader devices and user devices, which are configured to receive the continuously monitored analyte information. The CAM system includes a prediction model which predicts patient outcomes for use with a decision support model that can be implemented in different settings—such as a hospital setting, and a home monitoring setting—for the purpose of reducing patient readmission and providing guidance to support decisions made concerning patient health. For example, output of the decision support model can include alerts indicating recommended actions (e.g., treatment plan adjustment, discharge readiness, patient outreach, etc.), notifications about current and/or predicted conditions of the patient, and visual information (e.g., current and/or predicted trend graphs, current and/or predicted analyte levels). The visual information includes components that are displayed and, in some embodiments, dynamically updated for presentation based on patient information. Examples of such visual information include alerts, notifications, and recommendations for display and/or storage on user devices, patient monitoring devices (e.g., bedside monitors), and electronic medical records associated with the patient.

In some embodiments, the analyte being monitored includes lactate, glucose, ketones, creatinine or any combination of the foregoing. In some embodiments, the prediction model also bases the predicted patient outcomes on other medical information associated with the patient such as the patient's medical history, prior and current treatments, prior and current vital signs (e.g., blood pressure, heart rate, oxygen saturation, body temperature), diagnostic study results, trend information such as vital signs over time, changes in analyte levels, cardiac markers over time, and disease progression, and health care provider (HCP) notes and preferences. For reducing patient readmission, the decision support model of a CAM system may be further configured to provide alerts, notifications, and/or recommendations for treatment based on the predicted patient outcome, and in some embodiments, HCP preferences, and communication settings. Depending on the type of setting, such as a hospital or home, the alerts, notifications, and/or recommendations can be transmitted to different devices and recipients. In some embodiments, a patient can be associated with a sequence of multiple decision points within a decision matrix while the patient is still admitted or at discharge, such as if the patient is ready to move from the intensive care unit to the general recovery ward, or whether the patient is well enough to be discharged either to their home or an intermediary facility such as a nursing home or rehabilitation center. Accordingly, the decision support model includes generating multiple recommendations for a sequence of multiple decision points where the sequence of decision points can be based on the patient's condition and other medical attributes associated with the patient.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the arts to make and use the embodiments.

FIG. 1 is a diagram illustrating an operating environment of an example continuous analyte monitoring system for use with the techniques described herein.

FIG. 2 is a block diagram illustrating an example data receiving device for communicating with a sensor according to some embodiments.

FIG. 3 is a block diagram depicting an example analyte sensor device for communicating with a continuous analyte monitoring system according to some embodiments.

FIG. 4A is a diagram illustrating various operating environments of a continuous analyte monitoring system with a prediction model according to some embodiments.

FIG. 4B is a diagram illustrating various operating environments of a continuous analyte monitoring system with a distributed prediction model according to some embodiments.

FIG. 4C is a diagram illustrating the operating environment of the continuous analyte monitoring system in a hospital deployment according to some embodiments.

FIG. 5 is a flowchart illustrating a method for utilizing a continuous analyte monitoring system within a hospital environment for readmission reduction, according to some embodiments.

FIG. 6 is a flowchart illustrating a method for utilizing a continuous analyte monitoring system within a home environment for readmission reduction and reduction of avoidable admission, according to some embodiments.

FIG. 7 is a non-limiting exemplary table illustrating a method that utilizes a continuous lactate monitoring system to determine a proposed course of action for a patient. Both the concentration of lactate (level of lactate/lactate level) and rate of change (trend) of the lactate level of the patient are used to determine the appropriate proposed course of action for the patient.

FIG. 8 is a flowchart illustrating a method for utilizing a continuous analyte monitoring system in the detection of sepsis conditions, according to some embodiments.

FIG. 9 is a flowchart illustrating a method for utilizing a continuous analyte monitoring system during high-risk surgery procedures, according to some embodiments.

FIGS. 10A-C depict decision matrices within an early warning system, showing the relationship between trends in continuous analyte measurements, measured analyte levels, and corresponding clinical recommendations, according to some embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for an improved decision support system through the use of a continuous analyte monitoring system, a prediction model for generating potential patient outcomes based on the continuously monitored analyte levels, and a decision support model for generating alerts, notifications, and recommended actions based on the predicted patient outcomes. In some embodiments, the decision support system generates alerts, notifications, and recommendations for patient treatment decisions. In some embodiments, the alerts, notifications, and recommended actions provide insights for reducing readmission and providing guidance to support decisions made concerning patient health.

In embodiments for readmission reduction, the potential patient outcomes can be used to dynamically adjust alerts and notifications and direct patient treatment recommendations. In this disclosure, reducing readmission refers to implementing decision protocols to reduce rates in which a patient is readmitted into a hospital after a discharge. In some embodiments, the decision support model provides recommendations for a sequence of decisions, or a decision tree, for the patient, based on the patient's disease, condition, and other relevant medical attributes. Examples of a sequence of decisions includes decisions for moving the patient within the hospital setting, such as from the ICU to general recovery, and at discharge. The decision support model is trained to automatically determine the sequence of decisions for a particular patient and generate recommendations accordingly. In some embodiments, recommended actions include a recommendation to keep the patient admitted in the hospital (i.e., not discharged) for an additional period of time (e.g., for additional observation), a recommendation to send the patient to a subacute facility, or a time-sensitive recommendation for the patient to visit a medical professional, such as a primary care physician, when the patient has already been discharged from the hospital.

During acute illness or injury, levels of the hormone epinephrine are raised. This leads to the increased production of glucose through glycogenolysis and the subsequent conversion of glucose into pyruvate through aerobic glycolysis, which is later converted into lactate and results in increasing circulating levels of lactate. In addition, poor tissue oxygenation, most commonly due to hypovolemia and low blood pressure, may lead to anaerobic respiration which further contributes to rising lactate levels. Embodiments of the present disclosure utilize continuous lactate monitoring for measuring lactate levels. A prediction model can be updated in real-time based on the continuously monitored lactate information which improves the accuracy of the prediction of patient outcomes as well as the capability to dynamically recommend treatment adjustments. Increases in lactate levels are associated with worse clinical outcomes and early attention to, and awareness of, such changes can be used to direct treatment decisions in particular to reduce avoidable hospital readmissions. Continuous lactate monitoring can be used to monitor patients and screen for signs of deterioration. This reduces the blind spot of conventional decision support systems that rely on discrete patient observations by allowing for earlier identification of patient decline. In some embodiments, the decision support system utilizes the prediction model to generate relevant alerts, notifications, and recommendations for patient treatment based on the continuous lactate data. In some embodiments, the recommendations can be utilized as part of a readmission reduction process (e.g., a decision point for when to discharge a patient) that includes a decision matrix for the discharge process.

The present disclosure is not limited to lactate as the analyte being continuously monitored. Other analytes or measurements, in addition to or as alternatives to lactate, can also be monitored and/or utilized as part of the prediction model. These analytes and/or measurements include, but are not limited to, glucose, ketones, oxygen, hemoglobin A1C, or any combination thereof. The analytes being monitored may be based on the continuous analyte sensor, as discussed above, such as analyte sensors employing multiple enzymes for detection and in which multiple enzymes can function independently or in concert to detect one or more analytes, an electrochemical analyte sensor utilizing a lactate-responsive enzyme for lactate detection and quantification, and/or analyte sensors employing multiple enzymes for detection of multiple analytes and, more specifically, analyte sensors employing multiple working electrodes for detecting multiple analytes, e.g., glucose, β-hydroxybutyrate, uric acid, ketone, creatinine, ethanol, and lactate.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

As used herein and in the appended claims, the singular forms “a,” “an,” and “the” include plural referents unless the context clearly dictates otherwise. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20% (e.g., up to 10%, up to 5%, or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the term “continuous” as it relates to a continuous analyte sensor refers to a sensor that is configured to take one or more measurements of the analyte over a period of time. A continuous sensor can take sequential measurements according to its sampling frequency. For example, one or more measurements can be taken about every 1 ms, about every 10 ms, about every 100 ms, about every 1 s, about every 10 seconds, about every 30 seconds, about every minute, about every 5 minutes, about every 10 minutes, about every 30 minutes, or about every hour. The measurements can be taken continuously e.g. over a contiguous time period of at least 1 hour, 6 hours, 12 hours, 1 day, 2 days, 3 days, 4 days, 5 days, 6 days, 1 week, 2 weeks, 3 weeks, 1 month or longer. A continuous analyte sensor is typically continuously in contact with a sample, such as a biofluid. For example, a continuous analyte sensor can comprise an implantable portion or member which in use is in continuous contact with a biofluid such as dermal fluid or interstitial fluid, such that measurements can be taken continuously or periodically according to the sampling frequency of the sensor over the continuous time period.

As used herein, the term “measure” and variations thereof can encompass the meaning of a respective term, such as “determine,” “calculate,” and variations thereof.

As used herein, an “analyte” is an enzyme substrate that is subject to be measured or detected. The analyte can be from, for example, a biofluid and can be tested in vivo, ex vivo, or in vitro.

As used herein, a “sensor” is a device configured to detect the presence and/or measure the level of an analyte in a sample, including a continuous analyte sensor which is configured to detect analytes in the sample in a continuous manner.

The term “patient” refers to a living animal, and thus encompasses a living mammal and a living human, for example. The term “user” can be used herein as a term that encompasses the term “patient.”

As used herein, “readmission” refers to when a patient is admitted to a hospital again within a predetermined period of time after discharge from the hospital following an initial admission.

Generally, embodiments of the present disclosure include systems, devices, and methods for the use of in vivo analyte monitoring systems for multiple different use cases.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive. Sensors in the present disclosure are adapted to be at least partially inserted into a tissue of interest, such as within the dermal layer of the skin or in subcutaneous tissue. In some embodiments, the sensor can comprise a proximal portion configured to be positioned above a user's skin and a distal portion configured to be transcutaneously positioned through the user's skin and in contact with a bodily fluid. In some embodiments, the distal portion is configured to detect an analyte in the bodily fluid. In some embodiments, the proximal portion can be electrically coupled with processing electronics. In some embodiments, the processing electronics are disposed in the electronics housing of the sensor control device. The sensor can comprise a sensor of sufficient length for insertion to a desired depth in a given tissue. The sensor can comprise a sensing region or sensing area that is active for sensing lactate, and can comprise a lactate-responsive enzyme, according to one or more embodiments. The sensing region or sensing area can include a polymeric material to which the lactate-responsive enzyme is covalently bonded, according to some embodiments. In some embodiments of the present disclosure, lactate can be monitored in any biological fluid of interest such as dermal fluid, plasma, blood, lymph, synovial fluid, cerebrospinal fluid, saliva, bronchoalveolar lavage, amniotic fluid, or the like. In some embodiments, lactate-responsive sensors of the present disclosure can be adapted for interrogating dermal fluid or interstitial fluid.

An introducer can be present transiently to promote introduction of the sensor into a tissue. In illustrative embodiments, the introducer can comprise a needle. It is to be recognized that other types of introducers, such as sheaths or blades, can be present in alternative embodiments. More specifically, the needle or similar introducer can transiently reside in proximity to the sensor prior to insertion and then be withdrawn afterward. While present, the needle or other introducer can facilitate insertion of the sensor into a tissue by opening an access pathway for sensor to follow. For example, the needle can facilitate penetration of the epidermis as an access path way to the dermis to allow implantation of the sensor to take place, according to some embodiments. After opening the access pathway, the needle or other introducer can be withdrawn so that it does not represent a sharps hazard. In some embodiments, the needle can be solid or hollow, beveled or non-beveled, and/or circular or non-circular in cross-section. In some embodiments, the needle can be comparable in cross-sectional diameter and/or tip design to an acupuncture needle, which can have a cross-sectional diameter of about 250 microns, for example. It is to be recognized, however, that suitable needles can have a larger or smaller cross-sectional diameter if needed for particular applications.

In some embodiments, a tip of the needle can be angled over the terminus of the sensor, such that the needle penetrates a tissue first and opens an access pathway for the sensor. In some embodiments, the sensor can reside within a lumen or groove of the needle, with the needle similarly opening an access pathway for the sensor. In either case, the needle is subsequently withdrawn after facilitating insertion.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system, such as but not limited to wellness, fitness, dietary, research, information or any purposes involving analyte sensing over time. As used herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, or any combination thereof.

Before describing aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. CGM systems, for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (“NFC”) or Radio Frequency Identification (“RFID”) protocol. In vivo analyte monitoring systems can also operate without the need for finger stick calibration.

In vivo analyte monitoring systems can be differentiated from in vitro systems that contact a biological sample outside of the body (or ex vivo) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Exemplary Continuous Analyte Monitoring System

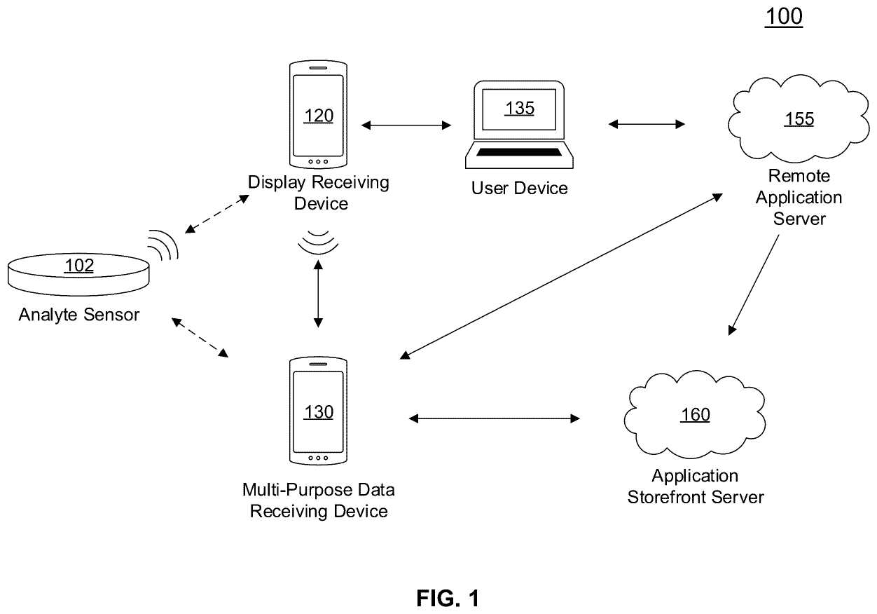

FIG. 1 illustrates an operating environment of a continuous analyte monitoring system 100 capable of embodying the techniques described herein. The continuous analyte monitoring system 100 can include a system of components designed to provide monitoring of parameters, such as analyte levels, of a human or animal body or can provide for other operations based on the configurations of the various components. As embodied herein, the system can include an analyte sensor 102, or simply “sensor” worn by the user or attached to the body for which information is being collected. As embodied herein, the analyte sensor 102 can be a sealed, disposable device with a predetermined active use lifetime (e.g., 1 day, 14 days, 30 days, etc.). Sensors 110 can be applied to the skin of the user body and remain adhered over the duration of the sensor lifetime or can be designed to be selectively removed and remain functional when reapplied. The low-power continuous analyte monitoring system 100 can further include a data reading device 120, multi-purpose data receiving device 130, and user device 135 configured as described herein to facilitate retrieval and delivery of data, including analyte data, from the analyte sensor 102.

One or more components of analyte system 100 can be physically located in one or more locations, such as the same location or distributed across different locations. For example, analyte sensor 102 is physically worn by the user who can be located at a home location or in a hospital setting; data receiving device 120 can be located in physical proximity to the analyte sensor 102 (e.g., such as in the same room as the analyte sensor 102) or in a separate physical location but in network communication with the analyte sensor 102. Similarly, other components of analyte system 100 can be located in physical proximity to the analyst sensor 102 or in a separate physical location.

As embodied herein, the continuous analyte monitoring system 100 can include a software or firmware library or application provided, for example via a remote application server 155 or application storefront server 160, to a third-party and incorporated into a multi-purpose data receiving device 130 such as a mobile phone, tablet, personal computing device, or other similar computing device capable of communicating with the analyte sensor 102 over a communication link. Multi-purpose hardware for multi-purpose data receiving device 130 can further include embedded devices, including, but not limited to fluid delivery systems or drug delivery systems (e.gb., insulin pumps or insulin pens), having an embedded library configured to communicate with the analyte sensor 102. For example, multi-purpose data receiving device 130 can be a mobile phone or tablet configured to communicate with analyte sensor 102 via an installed application or software program. When the application or software program are uninstalled, multi-purpose data receiving device 130 may be unable to communicate with analyte sensor 102. The installed application can configure multi-purpose data receiving device 130 so that it can process analyte data. Some examples of processing data include displaying the analyte data, analyzing the analyte data, and generating visual information representing the analyte data and associated alerts, notifications, and recommendations.

As embodied herein, the continuous analyte monitoring system 100 can further include a data receiving device 120 capable of communicating with analyte sensor 102 over a communication link and multi-purpose data receiving device 130. In contrast to multi-purpose data receiving device 130, data receiving device 120 can be a device dedicated (e.g., without having to install any additional application or software) to communicating with and processing data from analyte sensor 102. In other embodiments, data receiving device 120 is configured to communicate only with analyte sensor 102 and only process and display data received from and associated with analyte sensor 102.

As embodied herein, the continuous analyte monitoring system 100 can further include user device 135, which is configured to be in communication with data receiving device 120, multi-purpose data receiving device 130, and/or a remote application server 155. In some embodiments, user device 135 is implemented as a computer with a fixed location while data receiving device 120 and multi-purpose data receiving device 130 are implemented as mobile computers capable of being transported (e.g., with the patient, with a hospital bed). User device 135 can receive analyte data from data receiving device 120 and multi-purpose data receiving device 130 and provide additional output, input, and processing capabilities for interacting with the analyte data. For example, user device 135 can be implemented as a laptop or a personal computer that includes a keyboard, mouse, and/or a larger screen for displaying the analyte data.

One or more components of continuous analyte monitoring system 100 can be deployed within different physical settings, such as a hospital or home. Implementation of the components (e.g., data receiving device 120, multi-purpose data receiving device 130, user device 135) can be based on the setting in which continuous analyte monitoring system 100 is deployed. For example, in a hospital setting, one or more data receiving device 120 or multi-purpose data receiving device 130 can be implemented as a patient bedside monitoring device configured to receive and display analyte information provided by analyte sensor 102 and user device 135 can be implemented as a computing device located in a different physical location within the hospital, such as a nurse's station. Remote application server 155 can be configured with modules for storing and configuring electronic medical records (EMR) for patients in one or more hospitals. Remote application server 155 is configured to receive analyte information from user device 135 and/or data receiving device 120 and further configured to update EMR profiles associated with the received analyte information.

Communication between components is also based on the type of components. For example, the frequency of communicating analyte data between analyte sensor 102 and data receiving device 120 and/or user device 135 can be different from the frequency that the analyte data is communication to the remote application server. For example, data receiving device 120 and/or user device 135, when implemented in a hospital setting, can be utilized to display patient conditions in real-time; accordingly, the frequency of communication between analyte sensor 102 and data receiving device 120 and/or user device 135 can be configured to a high frequency to ensure that analyte data displayed by data receiving device 120 and/or user device 135 is accurate. On the other hand, the frequency of communication between data receiving device 120 and/or user device 135 and remote application server 155 can be set to a lower frequency (i.e., data is transmitted in batches) for storage at the remote application server 155.

Parameters of the communications between components can be dynamically updated based on any number of conditions including the component receiving the analyte data, the type of analyte being monitored, risk factors associated with the patient, time of day, current location of the patient, and a current condition of the patient. For example, configurable threshold settings associated with these conditions can trigger changes to the communication frequency. As one non-limiting example, a negative change in a patient's condition based on a certain threshold can trigger an increase in communication frequency. As another example, communications during evening hours can be set to a lower frequency then communications during daytime hours.

There can also be location-specific conditions for that dictate the type and frequency of communications. For example, the current location of the patient, such as whether the patient is located at home or within the hospital, can change the types and frequency of messages that are transmitted to the patient's caregiver. As one non-limiting example, when the user is determined to be at home, or otherwise physically located outside of a hospital, the analyte sensor 102 can be configured to communicate directly with remote application server 155 or the caregiver's device (e.g., multi-purpose data receiving device 130 or user device 135), such as when the user is located at home. In this manner, analyte information can be stored directly by the remote application server 155, such as in an EMR associated with the patient, and/or provided directly to the caregiver device even when the patient is located at his home.

Although the illustrated embodiments of the continuous analyte monitoring system 100 include only one of each of the illustrated devices, this disclosure contemplates the continuous analyte monitoring system 100 incorporate multiples of each components interacting throughout the system. For example and without limitation, as embodied herein, data reading device 120 and/or multi-purpose data receiving device 130 can include multiples of each. As embodied herein, multiple data receiving devices 130 can communicate directly with sensor 102 as described herein. Additionally or alternatively, a data receiving device 130 can communicate with secondary data receiving devices 130 to provide analyte data, or visualization or analysis of the data, for secondary display to the user or other authorized parties.

### Exemplary Reader Device

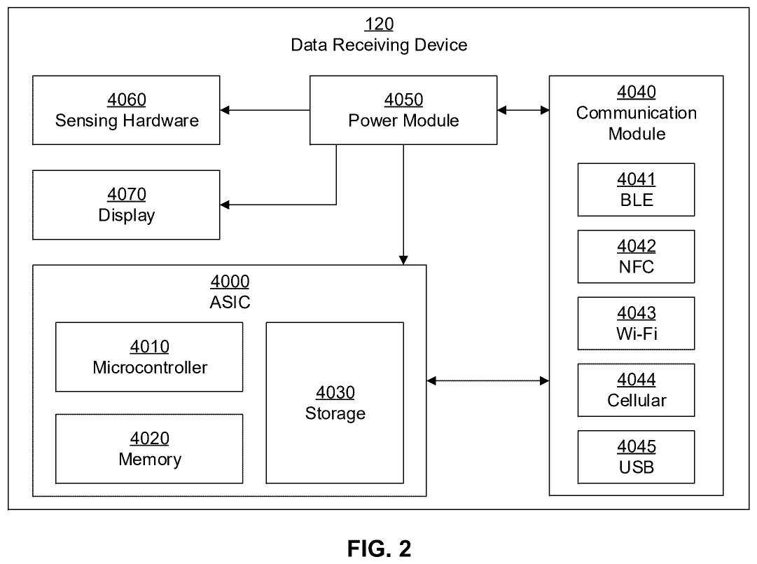

FIG. 2 is a block diagram depicting an example embodiment of a reader device configured as a smartphone or a patient bedside monitor. The following description will focus on data receiving device 120 but it is understood that the components and functionality can also apply to other reader devices, such as multi-purpose data receiving device 130 and user device 135. For purpose of illustration and not limitation, reference is made to the exemplary embodiment of data receiving device 120 for use with the disclosed subject matter as shown in FIG. 2. Data receiving device 120 and the related multi-purpose data receiving device 130 include components germane to the discussion of analyte sensor 102 and its operations and additional components can be included. In particular embodiments, data receiving device 120 and multi-purpose data receiving device 130 can be or include components provided by a third party and are not necessarily restricted to include devices made by the same manufacturer as analyte sensor 102.

As illustrated in FIG. 2, data receiving device 120 includes an ASIC 4000 having a microcontroller 4010, memory 4020, and storage 4030 and communicatively coupled with a communication module 4040. Power for the components of data receiving device 120 can be delivered by power module 4050, which as embodied herein can include a rechargeable battery. Data receiving device 120 can further include display 4070 for facilitating review of analyte data received from analyte sensor 102 or other device (e.g., user device 135 or remote application server 155). Data receiving device 120 can include separate user interface components (e.g., physical keys, light sensors, microphones, etc.).

Microcontroller 4010 can be implemented a general-purpose processing unit or processor (e.g., a central processing unit according to a Complex Instruction Set Computer (CISC) or Reduced Instruction Set Computer (RISC) architecture), a PGA or FPGA, a controller, and/or a custom processing architecture. Microcontroller 4014 can be centralized or distributed within electronics. For example, microcontroller 4014 can be centralized as a module within a single chip, distributed throughout a single chip, or distributed in one or more modules within a first ASIC chip and a second wireless communication electronics chip. The processing capability of microcontroller 4014 can be implemented in the various IC forms described herein using hardware, firmware, software, or a suitable combination of hardware, firmware, and software. Microcontroller 4014 can include a graphics processor unit (GPU) as a discrete component or integrated into a larger more comprehensive processor.

ASIC 4000 can further include memory 4020, which is non-transitory such as volatile and/or non-volatile memory for storing instructions, information, and collected data. Instructions are stored in memory 4020 in the form of software and/or firmware that is executable by microcontroller 4010. These instructions can be embedded, installed, or both embedded and installed in the electronics of ASIC 4000. Many types of memory can be used in data receiving device 120 including, but not limited to random access memory (e.g., DRAM, SRAM, SDRAM, DDR SDRAM, MRAM, RRAM), read only memory (e.g., flash memory, PROM, EPROM, EEPROM), variants thereof (e.g., virtual memory, register memory, cache memory), and/or others. Memory 4020 can be in the form of one or more discrete memory chips, can be distributed throughout the semiconductor chips of data receiving device 120, such as within a processor chip and within a wireless communications chip, or can be both in the form of one or more discrete chips and distributed throughout one or more chips.

In some embodiments, microcontroller 4014 can be implemented as a small ASIC designed to process analog front-end activities and communication module 4040, such as BLE module 4041 can be configured to implement complex processing and computing activity such as generating trend arrows, performing data smoothing, and performing calibration features.

Communication module 4040 can include Bluetooth low energy (BLE) module 4041 and NFC module 4042. Data receiving device 120 can be configured to wirelessly couple with analyte sensor 102 and transmit commands to and receive data from analyte sensor 102. As embodied herein, data receiving device 120 can be configured to operate, with respect to analyte sensor 102 as described herein, as an NFC scanner and a BLE end point via specific modules (e.g., BLE module 4042 or NFC module 4042) of communication module 4040. For example, data receiving device 120 can issue commands (e.g., activation commands for a data broadcast mode of the sensor; pairing commands to identify data receiving device 120) to analyte sensor 102 using a first module of the communication module 4040 and receive data from and transmit data to analyte sensor 102 using a second module of the communication module 4040.

As another example, communication module 4040 can include, for example, cellular radio module 4044. The cellular radio module 4044 can include one or more radio transceivers for communicating using broadband cellular networks, including, but not limited to third generation (“3G”), fourth generation (“4G”), and fifth generation (“5G”) networks. Additionally, communication module 4040 of data receiving device 120 can include Wi-Fi radio module 4043 for communication using a wireless local area network according to one or more of the IEEE 802.11 standards (e.g., 802.11a, 802.11b, 802.11 g, 802.11n (aka Wi-Fi 4), 802.11ac (aka Wi-Fi 5), 802.11ax (aka Wi-Fi 6)). Using cellular radio module 4044 or Wi-Fi radio module 4043, data receiving device 120 can communicate with remote application server 155 to receive analyte data or provide updates or input received from a user (e.g., through one or more user interfaces). Communication module 4040 can further include other protocol module 4045 that are compatible with additional wireless standards for Internet of Things (IoT) devices, such as Zigbee and Matter. In some embodiments, other protocol module 4045 can include additional or alternative chipsets for use with other short-range communication schemes, such as short-range radio (other than Bluetooth or BLE), personal area network according to IEEE 802.15 protocols, IEEE 802.11 protocols, infrared communications according to the Infrared Data Association standards (IrDA), etc.

In some embodiments, other protocol module 4045 can also be configured to be compatible with proprietary protocols such as proprietary short-range radio protocols that do not include Bluetooth or BLE. One non-limiting example is the Medical Implement Communication System (MICS) protocol which is configured for transmitting data between medical devices implanted in a patient. MICS protocol operates at a frequency from 402 to 405 MHz. Being compatible with other short-range radio protocols could be particular advantageous in a hospital setting where Bluetooth and/or BLE can potentially interfere with operations and communications with medical devices that are not intended recipients of Bluetooth and/or BLE messages. Other short-range radio protocols avoid this interference issue. Additionally, proprietary short-range radio protocols can also offer more security as Bluetooth and/or BLE can be susceptible to cyberattacks.

As embodied herein, on-board storage 4030 of data receiving device 120 can store analyte data received from analyte sensor 102. Further, data receiving device 120, multi-purpose data receiving device 130, or user device 135 can be configured to communicate with remote application server 155 via a wide area network. As embodied herein, analyte sensor 102 can provide data to data receiving device 120 or multi-purpose data receiving device 130. Data receiving device 120 can transmit the data to user device 135. User device 135 (or multi-purpose data receiving device 130) can in turn transmit that data to remote application server 155 for processing and analysis.

As embodied herein, data receiving device 120 can further include sensing hardware 4060 similar to, or expanded from, sensing hardware 5060 of analyte sensor 102. In particular embodiments, data receiving device 120 can be configured to operate in coordination with analyte sensor 102 and based on analyte data received from the analyte sensor 102. As an example, where analyte sensor 102 is a glucose sensor, data receiving device 120 can be or include an insulin pump or insulin injection pen. In coordination, multi-purpose data receiving device 130 can adjust an insulin dosage for a user based on glucose values received from the analyte sensor.

In some embodiments, data receiving device 120 can also include wired communication electronics for communicating according to a wired protocol with user device 135 and remote application server 155. Examples of such wired protocols include Universal Serial Bus (USB), Universal Asynchronous Receiver Transmitter (UART), Universal Synchronous Asynchronous Receiver Transmitter (USART), RS-232, RS-485, and/or ethernet. USB connections can be authenticated on each plug event. Authentication can use, for example, a two-, three-, four-, or five-pass design with different keys. The USB system can support a variety of different sets of keys for encryption and authentication. Keys can be aligned with differential roles (clinical, manufacturer, user, etc.). Sensitive commands that can leak security information can trigger authenticated encryption using an authenticated additional keyset.

### Exemplary Sensor Devices

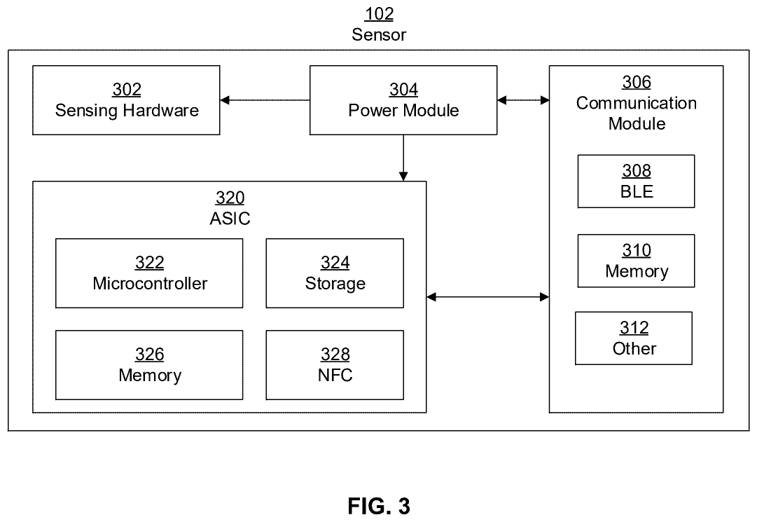

FIG. 3 is a block diagram depicting example embodiments of sensor 102. For purpose of illustration and not limitation, reference is made to the exemplary embodiment of analyte sensor 102 for use with the disclosed subject matter as shown in FIG. 3. FIG. 3 illustrates a block diagram of an example analyte sensor 102 according to exemplary embodiments compatible with the security architecture and communication schemes described herein.

Sensor 102 can be used to monitor other analytes and can be configured as discussed above to detect one or more analytes. The monitored analyte can be selected from alcohol, cardiac markers, creatine kinase (e.g., CK-MB), creatine, ketone, lactate, oxygen, potassium, and/or prostate-specific antigen. One or more analytes are monitored by a given sensor 102 or combination of sensors 102. In some embodiments, the monitored analyte is lactate. In some embodiments, the monitored analytes is a combination of glucose and lactate.

The term sensor 102 can collectively refer to partially implantable sensors, fully implantable sensors, wholly ex vivo sensors, or a combination of any of the above. When implemented as a partially implantable sensor, sensor 102 can have a first end portion that is inserted into the user's body into contact with bodily fluid (e.g., interstitial fluid, dermal fluid, and/or blood) to sense the analyte level while placed in the body. An opposite end portion of sensor 102 is held by electronics within sensor 102. Partially implantable sensors can be placed in the user's body using an applicator that can be provided (e.g., sold) with an on-body device (OBD). In some embodiments, the OBD is pre-loaded inside an applicator prior to inserting sensor 102. In some embodiments, the applicator includes a pre-loaded OBD with a pre-connected sensor. Either embodiment can be done by the manufacturer or user, but in any event done before sensor insertion. When performed by a user, the user can perform this procedure in any suitable location when convenient without the aid of a medical professional.

In some embodiments, sensor 102 is configured to be inserted in the body of the user such that a sensing part of sensor 102 is under the skin, in a subcutaneous location. More specifically, a part of sensor 102 is placed under the skin (e.g., the sensing part that is configured to measure the analyte level). Thus, sensor 102 is configured to extend through the skin. In other words, sensor 102 can be referred to as a transcutaneous sensor. Sensor 102 is configured to be in contact with bodily fluid. In this embodiment, sensor 102 is configured to be in contact with interstitial fluid. In other examples, the bodily fluid can be blood from a capillary. In other examples, sensor 102 can be configured to be inserted into the skin such that the sensing part resides in the epidermis or more preferably the dermis (the dermal layer). Thus, the bodily fluid can be dermal fluid. Sensor 102 can be inserted, and OBD applied, by the user or another user to the user, for example by using an inserter or applicator.

In some embodiments, sensor 102 can be implemented as an electrochemical sensor. For example, sensor 102 can be configured to sample the analyte level of the user and output a signal (e.g., a current or voltage) for receipt by electronics. Sensor 102 can be configured to sample the analyte level continuously, at random intervals, at periodic intervals (where each sampling is a discrete single sampling or a sampling for a longer duration), and/or on-demand.

As embodied herein, analyte sensor 102 can include ASIC 320 communicatively coupled with a communication module 306. ASIC 320 can include a microcontroller core 322, on-board memory 326, and storage memory 324. Storage memory 324 can store data used in an authentication and encryption security architecture. Storage memory 324 can store programming instructions for analyte sensor 102. As embodied herein, certain communication chipsets can be embedded in the ASIC 320 (e.g., an NFC transceiver 328). ASIC 320 can receive power from a power module 5050, such as an on-board battery or from an NFC pulse. Storage memory 324 of the ASIC 320 can be programmed to include information such as an identifier for analyte sensor 102 for identification and tracking purposes. Storage memory 324 can also be programmed with configuration or calibration parameters for use by analyte sensor 102 and its various components. Storage memory 324 can include rewritable or one-time programming (“OTP”) memory. The storage memory 324 can be updated using techniques described herein to extend the usefulness of analyte sensor 102.

To perform its functionalities, sensor 102 can further include suitable sensing hardware 302 appropriate to its function. As embodied herein, sensing hardware 302 can include an analyte sensor transcutaneously, intradermally, or subcutaneously positioned in contact with a bodily fluid of a subject. The analyte sensor can generate sensor data containing values corresponding to levels of one or more analytes within the bodily fluid.

The storage memory 324 of the sensor 102 can include the software blocks related to communication protocols of the communication module. For example, the storage memory 324 can include a BLE services software block with functions to provide interfaces to make the BLE module 308 available to the computing hardware of the sensor 102. These software functions can include a BLE logical interface and interface parser. BLE services offered by the communication module 306 can include the generic access profile service, the generic attribute service, generic access service, device information service, data transmission services, and security services. The data transmission service can be a primary service used for transmitting data such as sensor control data, sensor status data, analyte measurement data (historical and current), and event log data. The sensor status data can include error data, current time active, and software state. The analyte measurement data can include information such as current and historical raw measurement values, current and historical values after processing using an appropriate algorithm or model, projections and trends of measurement levels, comparisons of other values to patient-specific averages, calls to action as determined by the algorithms or models and other similar types of data.

As embodied herein, communication module 306 of sensor 102 can be implemented as or include one or more modules to support analyte sensor 102 communicating with other devices of the continuous analyte monitoring system 100. As an example only and not by way of limitation, example communication module 306 can include BLE module 308, memory 310, and other protocol module 312. As used throughout this disclosure, BLE refers to a short-range communication protocol optimized to make pairing of Bluetooth devices simple for end users. Communication module 306 can transmit and receive data and commands via interaction with similarly capable communication modules of data receiving device 120 or user device 135. Communication module 306 can store the data and commands in memory 310. Communication module 306 can include an additional module 312 for other protocols and/or additional or alternative chipsets for use with similar short-range communication schemes, such as a personal area network according to IEEE 802.15 protocols, IEEE 802.11 protocols, infrared communications according to the Infrared Data Association standards (IrDA), etc.

According to aspects of the disclosed subject matter, and as embodied herein, a sensor 102 can be configured to communicate with multiple devices concurrently by adapting the features of a communication protocol or medium supported by the hardware and radios of the sensor 102. As an example, the BLE module 308 of the communication module 306 can be provided with software or firmware to enable multiple concurrent connections between the sensor 102 as a central device and the other devices such as anyone of data receiving device 120, multi-purpose data receiving device 130, and/or user device 135.

Connections, and ensuing communication sessions, between two devices using a communication protocol such as BLE can be characterized by a similar physical channel operated between the two devices (e.g., a sensor 102 and data receiving device 120). The physical channel can include a single channel or a series of channels, including for example and without limitation using an agreed upon series of channels determined by a common clock and channel- or frequency-hopping sequence. Communication sessions can use a similar amount of the available communication spectrum, and multiple such communication sessions can exist in proximity. In certain embodiment, each collection of devices in a communication session uses a different physical channel or series of channels, to manage interference of devices in the same proximity.

For purpose of illustration and not limitation, reference is made to an exemplary embodiment of a procedure for a sensor-receiver connection for use with the disclosed subject matter. First, the sensor 102 repeatedly advertises its connection information to its environment in a search for a data receiving device 120. The sensor 102 can repeat advertising on a regular basis until a connection established. The data receiving device 120 detects the advertising packet and scans and filters for the sensor 102 to connect to through the data provided in the advertising packet. Next, data receiving device 120 sends a scan request command and the sensor 102 responds with a scan response packet providing additional details. Then, the data receiving device 120 sends a connection request using the Bluetooth device address associated with the data receiving device 120. The data receiving device 120 can also continuously request to establish a connection to a sensor 102 with a specific Bluetooth device address. Then, the devices establish an initial connection allowing them to begin to exchange data. The devices begin a process to initialize data exchange services and perform a mutual authentication procedure.

During a first connection between the sensor 102 and data receiving device 120, the data receiving device 120 can initialize a service, characteristic, and attribute discovery procedure. The data receiving device 120 can evaluate these features of the sensor 102 and store them for use during subsequent connections. Next, the devices enable a notification for a customized security service used for mutual authentication of the sensor 102 and data receiving device 120. The mutual authentication procedure can be automated and require no user interaction. Following the successful completion of the mutual authentication procedure, the sensor 102 sends a connection parameter update to request the data receiving device 120 to use connection parameter settings preferred by the sensor 102 and configured to maximum longevity.

In some embodiments, the data receiving device 120 then performs sensor control procedures to backfill historical data, current data, event log, and factory data. As an example, for each type of data, the data receiving device 120 sends a request to initiate a backfill process. The request can specify a range of records defined based on, for example, the measurement value, timestamp, or similar, as appropriate. The sensor 102 responds with requested data until all previously unsent data in the memory of the sensor 102 is delivered to the data receiving device 120. The sensor 102 can respond to a backfill request from the data receiving device 120 that all data has already been sent. Once backfill is completed, the data receiving device 120 can notify sensor 102 that it is ready to receive regular measurement readings. The sensor 102 can send readings across multiple notifications result on a repeating basis. As embodied herein, the multiple notifications can be redundant notifications to ensure that data is transmitted correctly. Alternatively, multiple notifications can make up a single payload.

As embodied herein, certain calibration features for the sensing hardware 302 of the analyte sensor 102 can be adjusted based on external or interval environment features as well as to compensate for the decay of the sensing hardware 302 during expended period of disuse (e.g., a “shelf time” prior to use). The calibration features of the sensing hardware 302 can be autonomously adjusted by the sensor 102 (e.g., by operation of the ASIC 320 to modify features in the memory 326 or storage memory 324) or can be adjusted by other devices of the CAM system 100.

As an example, sensor sensitivity of the sensing hardware 302 can be adjusted based on external temperature data or the time since manufacture. When external temperatures are monitored during the storage of the sensors, the disclosed subject matter can adaptively change the compensation to sensor sensitivity over time when the device experiences changing storage conditions. For purpose of illustration not limitations, adaptive sensitivity adjustment can be performed in an “active” storage mode where the analyte sensor 102 wakes up periodically to measure temperature. These features can save the battery of the analyte device and extend the lifespan of the analyte sensors. At each temperature measurement, the analyte sensor 102 can calculate a sensitivity adjustment for that time period based on the measured temperature. Then, the temperature-weighted adjustments can be accumulated over the active storage mode period to calculate a total sensor sensitivity adjustment value at the end of the active storage mode (e.g., at insertion). Similarly, at insertion, the analyte sensor 102 can determine the time difference between manufacture of the sensor 102 (which can be written to the storage memory 324 of the ASIC 320) or the sensing hardware 302 and modify sensor sensitivity or other calibration features according to one or more known decay rates or formulas.

Additionally, for purpose of illustration and not limitation, as embodied herein, sensor sensitivity adjustments can account for other sensor conditions, such as sensor drift. Sensor sensitivity adjustments can be hardcoded into the analyte sensor 102 during manufacture, for example in the case of sensor drift, based on an estimate of how much an average sensor would drift. Analyte sensor 102 can use a calibration function that has time-varying functions for sensor offset and gain, which can account for drift over a wear period of the sensor. Thus, analyte sensor 102 can utilize a function used to transform an interstitial current to interstitial glucose utilizing device-dependent functions describing analyte sensor 102 drift over time, and which can represent sensor sensitivity, and can be device specific, combined with a baseline of the glucose profile. Such functions to account for sensor sensitivity and drift can improve analyte sensor 102 accuracy over a wear period and without involving user calibration.

In some embodiments, the sensor 102 detects raw measurement values from sensing hardware 302. On-sensor processing can be performed, such as by one or more models trained to interpret the raw measurement values. Models can be machine learned models trained off-device to detect, predict, or interpret the raw measurement values to detect, predict, or interpret the levels of one or more analytes. Additional trained models can operate on the output of the machine learning models trained to interact with raw measurement values. As an example, models can be used to detect, predict, or recommend events based on the raw measurements and type of analyte(s) detected by the sensing hardware 302. Events can include, initiation or completion of physical activity, meals, application of medical treatment or medication, emergent health events, and other events of a similar nature.

Models can be provided to any combination of the sensor 102, data receiving device 120, or multi-purpose data receiving device 130 during manufacture or during firmware or software updates. Models can be periodically refined, such as by the manufacturer of the sensor 102 or the operator of the CAM system 100, based on data received from the sensor 102 and data receiving devices of an individual user or multiple users collectively. In certain embodiments, the sensor 102 includes sufficient computational components to assist with further training or refinement of the machine learned models, such as based on unique features of the user to which the sensor 102 is attached. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The models can also include algorithmic or rules-based models in addition to machine learned models. Model-based processing can be performed by other devices, including the data receiving device 120 or multi-purpose data receiving device 130, upon receiving data from the sensor 102 (or other downstream devices).

When applied to continuous lactate monitoring, machine learning models can rely on the real-time data being provided by, for example, sensor 102, to continuously train the model or update the training set for the model. Machine learning models of the present disclosure can also be trained using patient vital information, patient medical history, and in some embodiments, cohort information, which would increase the size of the training set to include lactate and medical information from patients that are similar to the current patient.

Data transmitted between the sensor 102 and any one of a data receiving device 120, multi-purpose data receiving device 130, and/or user device 135 can include raw or processed measurement values. Data transmitted between the sensor 102 and any one of a data receiving device 120, multi-purpose data receiving device 130, and/or user device 135 can further include alarms or notification for display to a user. The data receiving device 120, multi-purpose data receiving device 130, and/or user device 135 can display or otherwise convey notifications to the user based on the raw or processed measurement values or can display alarms when received from the sensor 110. Alarms that can be triggered for display to the user include alarms based on the predicted patient outcome (e.g., when compared to different levels of severity thresholds), direct analyte values (e.g., one-time reading exceeding a threshold or failing to satisfy a threshold), analyte value trends (e.g., average reading over a set period of time exceeding a threshold or failing to satisfy a threshold; slope); analyte value predictions (e.g., algorithmic calculation based on analyte values exceeds a threshold or fails to satisfy a threshold), sensor alerts (e.g., suspected malfunction detected), communication alerts (e.g., no communication between sensor 102 and data receiving device 120 for a threshold period of time; unknown device attempting or failing to initiate a communication session with the sensor 102), reminders (e.g., reminder to charge data receiving device 120; reminder to take a medication or perform other activity), and other alerts of a similar nature. For purpose of illustration and not limitation, as embodied herein, the alarm parameters described herein can be configurable by a user or can be fixed during manufacture, or combinations of user-settable and non-user-settable parameters.

### Exemplary Over-the-Air Updates

FIG. 1 further illustrates an example operating environment for providing over-the-air (“OTA”) updates for use with the techniques described herein. An operator of continuous analyte monitoring system 100 can bundle updates for data receiving device 120 or analyte sensor 102 into updates for an application executing on multi-purpose data receiving device 130. Using available communication channels between data receiving device 120, multi-purpose data receiving device 130, and analyte sensor 102, multi-purpose data receiving device 130 can receive regular updates for data receiving device 120 or analyte sensor 102 and initiate installation of the updates on data receiving device 120 or analyte sensor 102. Multi-purpose data receiving device 130 acts as an installation or update platform for data receiving device 120 or analyte sensor 102 because the application that enables the multi-purpose data receiving device 130 to communicate with analyte sensor 102, data receiving device 120 and/or remote application server 155 can update software or firmware on data receiving device 120 or analyte sensor 102 without wide-area networking capabilities.

As embodied herein, remote application server 155 operated by the manufacturer of analyte sensor 102 and/or the operator of continuous analyte monitoring system 100 can provide software and firmware updates to the devices of continuous analyte monitoring system 100. In particular embodiments, remote application server 155 can provides the updated software and firmware to user device 135 or directly to a multi-purpose data receiving device. As embodied herein, remote application server 155 can also provide application software updates to application storefront server 160 using interfaces provided by the application storefront. Multi-purpose data receiving device 130 can contact the application storefront server 160 periodically to download and install the updates.

After multi-purpose data receiving device 130 downloads an application update including a firmware or software update for data receiving device 120 or analyte sensor 102, data receiving device 120 or analyte sensor 102 and multi-purpose data receiving device 130 establish a connection. Multi-purpose data receiving device 130 determines that a firmware or software update is available for data receiving device 120 or analyte sensor 102. Multi-purpose data receiving device 130 can prepare the software or firmware update for delivery to data receiving device 120 or analyte sensor 102. As an example, multi-purpose data receiving device 130 can compress or segment the data associated with the software or firmware update, can encrypt or decrypt the firmware or software update, or can perform an integrity check of the firmware or software update. Multi-purpose data receiving device 130 sends the data for the firmware or software update to data receiving device 120 or analyte sensor 102. Multi-purpose data receiving device 130 can also send a command to data receiving device 120 or analyte sensor 102 to initiate the update. Additionally or alternatively, multi-purpose data receiving device 130 can provide a notification to the user of multi-purpose data receiving device 130 and include instructions for facilitating the update, such as instructions to keep data receiving device 120 and multi-purpose data receiving device 130 connected to a power source and in close proximity until the update is complete.

Data receiving device 120 or analyte sensor 102 receives the data for the update and the command to initiate the update from multi-purpose data receiving device 130. Data receiving device 120 can then install the firmware or software update. To install the update, data receiving device 120 or analyte sensor 102 can place or restart itself in a so-called “safe” mode with limited operational capabilities. Once the update is completed, data receiving device 120 or analyte sensor 102 re-enters or resets into a standard operational mode. Data receiving device 120 or analyte sensor 102 can perform one or more self-tests to determine that the firmware or software update was installed successfully. Multi-purpose data receiving device 130 can receive the notification of the successful update. Multi-purpose data receiving device 130 can then report a confirmation of the successful update to remote application server 155.

In particular embodiments, storage memory 324 of analyte sensor 102 includes OTP memory. The term OTP memory can refer to memory that includes access restrictions and security to facilitate writing to particular addresses or segments in the memory a predetermined number of times. Storage memory 324 can be prearranged into multiple pre-allocated memory blocks or containers. The containers are pre-allocated into a fixed size. If storage memory 324 is OTP memory, the containers can be considered to be in a non-programmable state. Additional containers which have not yet been written to can be placed into a programmable or writable state. Containerizing storage memory 324 in this fashion can improve the transportability of code and data to be written to storage memory 324. Updating the software of a device (e.g., the sensor device described herein) stored in an OTP memory can be performed by superseding only the code in a particular previously written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

### Summary of Various Continuous Analyte Monitoring System Deployments

The following figures depict an early warning system for generating visual information, including alerts, notifications, and visual content based on continuous analyte data. Visual information and visualizations are used synonymously in this disclosure. In some embodiments, the visualizations can be utilized as part of a readmission reduction process for a decision matrix for making decisions associated with a patient.

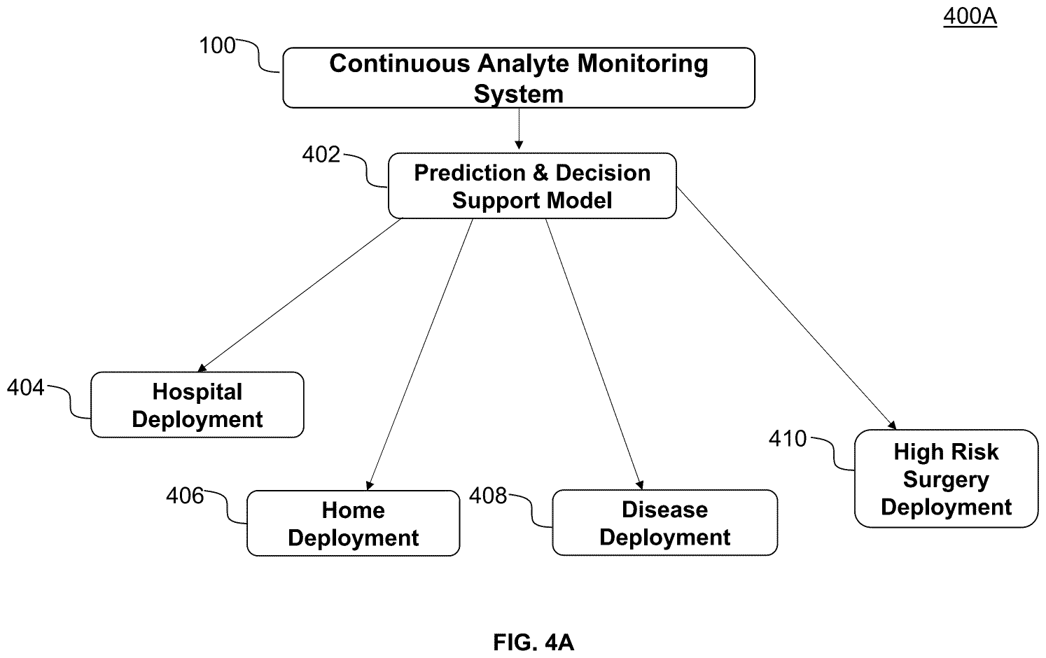

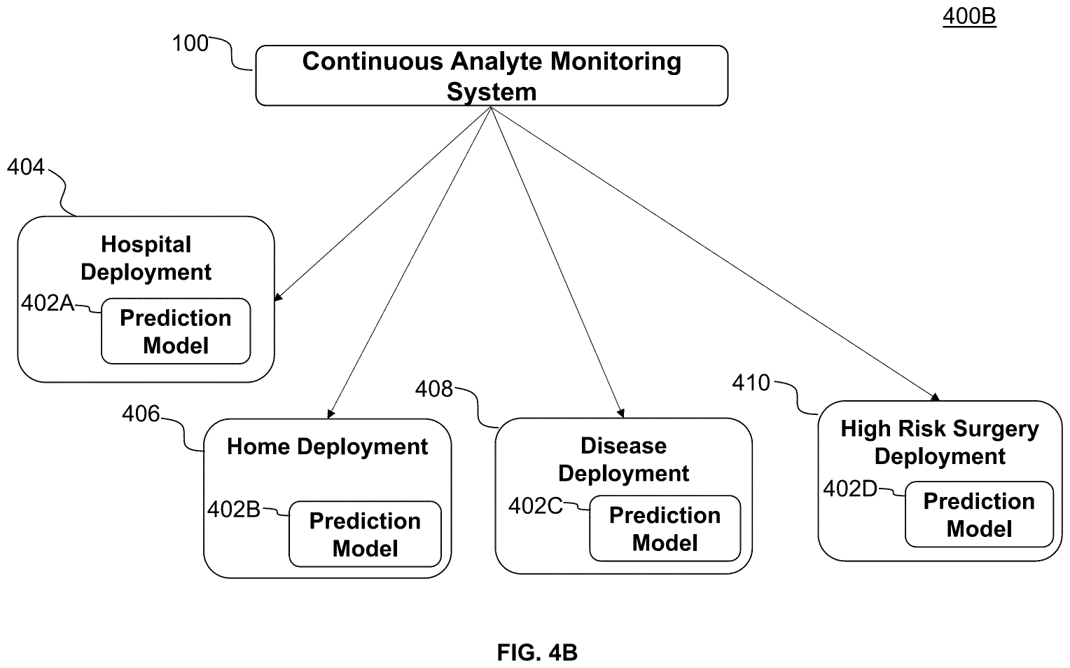

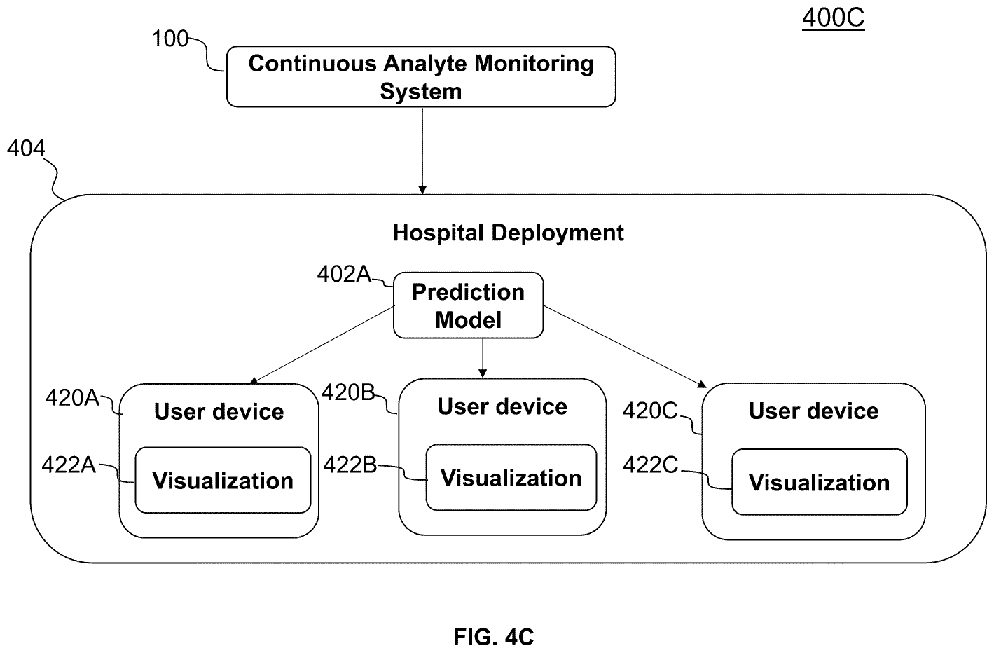

FIG. 4A is a block diagram of an early warning system 400A that includes continuous analyte monitoring (CAM) system 100 that can be deployed in various operating environments including one or more of a hospital deployment 404, home deployment 406, disease deployment 408, and high-risk surgery deployment 410, according to some embodiments.

CAM system 100 continuously monitors analyte levels of patients and provide the analyte information to a prediction and decision support model 402, which is configured to process the analyte information and provide predictions for use in various clinical settings, such as one or more of hospital deployment 404, home deployment 406, disease deployment 408, or high-risk surgery deployment 410. Prediction and decision support model 402 (herein after “prediction model 402”) is not limited to only generating predictions of patient outcomes but can also generate recommended actions or treatments for treating the predicted conditions. Examples of actions or treatments include a decision whether to discharge the patient. In some embodiments, the predictions and/or the recommended treatments can be transmitted and displayed on one or more of data receiving device 120, multi-purpose data receiving device 130, user device 135 and/or remote application server 155. In some embodiments, prediction model 402 provides the predictions to CAM system 100 for dynamic and automatic execution of treatment, such as by transmitting commands to treatment devices connected to CAM system 100. As another example, a predicted future analyte level is used to determine a recommendation for a medication dosage. In some embodiments, the predictions and/or the recommended treatments are transmitted and displayed on one or more of data receiving device 120, multi-purpose data receiving device 130, user device 135 and/or remote application server 155. In some embodiments, prediction model 402 provides the predictions to CAM system 100 for dynamic and automatic execution of treatment, such as by transmitting commands to treatment devices connected to CAM system 100.

Although only one prediction model 402 is depicted in FIG. 4, CAM system 100 can utilize more than one prediction model. For example, FIG. 4B depicts an early warning system 400B that includes a prediction model 402 generated and trained for each type of deployment, with prediction model 402A dedicated for generating predictions for hospital deployment 404, prediction model 402B for generating predictions for home deployment 406, prediction model 402C for generating predictions for disease detection deployment 408, and prediction model 402D for high-risk surgeries deployment 410. Each prediction model can be configured to provide predictions for patient outcomes based on analyte data for a patient.

In some embodiments, there is a prediction model 402 generated and trained for each deployment, i.e., a prediction model 402 dedicated for generating predictions and actions for hospital deployments, one for generating predictions and actions for home deployments. Prediction models are configured to provide predictions for patient outcomes based on analyte information for a patient and to provide recommended actions to facilitate treatment plans for the patient which can include whether to continue treatment of the patient in the hospital, whether to discharge the patient, to where the patient should be discharge (e.g., home, intermediate locations), and/or whether the patient should see a health care professional (e.g., when the patient has already been discharged).

In some embodiments, prediction model 402A implemented in a hospital deployment 404 can include one or more prediction models implemented at respective hospital locations, such that prediction model 402A can be trained on data specific to each hospital and therefore provide predictions that are more specific to each hospital to which the prediction model 402A is deployed. Further still, in some embodiments, prediction model 402A can comprise multiple prediction models within hospital deployment 404, with each prediction model being implemented on a per-patient (or per-department) basis within hospital deployment 404. Each prediction model can be trained on data specific to each patient (or department) to provide predictions that are more specific to each patient (or department) within the hospital deployment 404.

Prediction model 402B can be implemented on a per-patient basis (e.g., installed on a device associated with the patient such as data receiving device 120, multi-purpose data receiving device 130, and/or user device 135) and trained with data specific to each patient and to the home deployment 406. Prediction model 402B may therefore differ in its training than prediction model 402A, and is trained to generate predictions that are more tailored for each patient and the home deployment 406.

Prediction model 402C can be implemented on a per-disease basis (e.g., sepsis, heart failure) and trained with data specific to each disease within disease deployment 408. Prediction model 402C can therefore be trained to generate predictions that are more tailored for each disease. Prediction model 402D can be implemented for specific high-risk surgeries and trained to generate predictions that are more tailored for each surgery.

In another example embodiment, artificial intelligence (AI), such as machine-learning (ML) systems, train prediction model 402 to generate more accurate predictions of patient outcomes based on analyte data and other patient information provided by other medical devices associated with patients and more accurate recommended actions for the purpose of reducing readmission. The prediction model can be continuously updated with actual patient outcomes to determine the accuracy of outcome predictions. ML involves computers discovering how they can perform tasks without being explicitly programmed to do so. ML includes, but is not limited to, deep learning, fuzzy learning, supervised learning, unsupervised learning, etc. Machine learning algorithms can build an initial prediction model based on sample data, known as “training data,” in order to make predictions or decisions without being explicitly programmed to do so. This sample data typically includes analyte data from any number of patients and can be provided by any number of CAM systems. For supervised learning, the computer is presented with example inputs and their desired outputs with the computer's goal being to learn a general rule that maps inputs to outputs. In another example, for unsupervised learning, no labels are given to the learning algorithm, leaving it on its own to find structure in its input. Unsupervised learning can be the ultimate goal such as discovering trends or patterns in data, or a means towards another goal, such as improved accuracy of future predictions.

A machine-learning engine can use various classifiers to map concepts or data provided by different CAM systems and/or different deployment to capture relationships between concepts (e.g., analyte levels and patient outcomes in different deployments) and an accuracy of prior predicted patient outcomes. The classifier (discriminator) is trained to distinguish (recognize) in variations of data from different patients, CAM systems, and/or different deployments.

In some aspects, machine learning models are trained on a remote machine learning platform using a history of analyte data and patient information from one or more other patients, CAM systems, and/or different deployments. In addition, collecting patient data into large training sets can allow for the training data to be normalized (e.g., not skewed by a single or few occurrences of a data artifact). In one embodiment, prediction models are continuously updated as new patient information is received.

Hospital deployment 404 refers to utilizing analyte information (e.g., continuous lactate and/or glucose information) from a patient to predict the patient's medical outcome and to provide recommended actions to facilitate treatment plans for the patient when the patient is located within a hospital setting. Hospital deployment 404 can be used for the purposes of determining if a patient should be discharged or held at the hospital for continued treatment and/or observation. Hospital deployment 404 can additionally be used for the purposes of determining a preferred patient discharge setting, e.g. home or a rehabilitation facility. In this manner, hospital deployment 404 can be used to reduce readmission. A CAM system 100 implemented within a hospital deployment 404 can be configured with devices that are typically utilized within the hospital setting. For example, any one of data receiving device 120, multi-purpose data receiving device 130, and user device 135 can be implemented as any one of a patient bedside monitoring device, one or more computers at a nursing station, and one or more handheld devices carried by caregivers in the hospital setting. In some embodiments, any number and combination of data receiving device 120, multi-purpose data receiving device 130, and user device 135 can be implemented as a patient bedside monitoring ecosystem with each device implemented in the ecosystem capable of transmitting and receiving analyte data and other patient data from other devices. Devices in the patient bedside monitoring ecosystem can be configured to synchronize data with each other. Additionally, a CAM system 100 implemented within a hospital deployment 404 can be configured with to communicate with a sensor, such as an accelerometer, or similar, to monitor physical conditions of the patient, such as movement. Prediction models can be configured to determine whether physical exertion is a potential reason for elevated analyte levels (i.e., lactate). For example, prediction models can correlate the timing between monitored physical activity (e.g., based on data provided by an accelerometer) to analyte levels and other user activity, such as meals.

Home deployment 406 refers to utilizing analyte information (e.g., continuous lactate and/or glucose information) from a patient to predict the patient's medical outcome and to provide recommended actions to facilitate treatment plans for the patient when the patient is located within a home setting. Home deployment 406 can be used for the purposes of determining if a patient should be readmitted to the hospital or see a health care professional, or if a course of action not necessitating readmission to the hospital should be taken. In this manner, home deployment 406 can be used to reduce readmission and provide earlier care to those that require readmission. A CAM system 100 implemented within a home deployment 406 can be configured with devices that are typically utilized within the home setting. For example, any one of data receiving device 120, multi-purpose data receiving device 130, and user device 135 can be implemented as any one of a patient bedside monitoring device and one or more handheld devices carried by caregivers of the patient for receiving updates about their patients. Additionally, a CAM system 100 implemented within a home deployment 406 can be configured with a motion sensor, such as an accelerometer, or similar. As noted above, data from motion sensor can be utilized to determine whether increased analyte levels are caused by physical exertion, increasing the accuracy of the predictions from the prediction model.

Disease deployment 408 refers to utilizing analyte data (e.g., continuous lactate and/or glucose information) from a patient to predict the patient's medical outcome for different disease detection use cases, including sepsis and heart failure.

High risk surgery deployment 410 refers to utilizing analyte data (e.g., continuous lactate and/or glucose information) from a patient to predict the patient's medical outcome when the patient has undergone high-risk surgeries.

These deployments are discussed further below. In each of the deployments, CAM system 100 and a trained prediction model (e.g., prediction model 402, or prediction models 402A-D) can be used to identify trigger conditions for transmitting alerts and generating visualizations representing one or more of current status of the patient, current monitored analyte levels, current vital sign information, and proposed adjustments to a current treatment regime.

Continuous analyte data is provided to the prediction model to provide for continuous and real-time prediction of patient condition. Accordingly, in contrast to conventional early warning score systems and decision support systems, prediction models of the present disclosure can more accurately predict patient conditions, more quickly identify trigger conditions based on the predicted patient conditions, and more efficiently alert or notify appropriate recipients with recommended courses of action. Examples of such recommend courses of action include adjustments to a patient's treatment for the particular disease. For example, for a disease deployment 408 involving heart failure, possible adjustments to the patient's treatment can include increased monitoring, updated medication plan, providing mechanical or electrical support to the heart, such as implantation of an LVAD, pacemaker, defibrillator, or other cardiac assist device, and recommending a heart transplant.

As will be discussed further below, prediction models of the present disclosure can further be trained to utilize additional patient health information in addition to continuous analyte information as part of generating predictions for patient conditions. Examples of additional patient health information include vital signs such as heart rate, blood pressure, temperature, and oxygen levels, as well as patient activity levels such as number of steps and movement. The prediction models described herein can be trained to identify a combination of patient information that is most relevant for generating predictions for the patient condition. For example, prediction models can utilize a first set of patient information for a patient that has heart failure and a second set of patient information for a patient that has sepsis.

In all deployments, output (e.g., predicted patients conditions and visualizations, which can include alerts, notifications, and visual content, or a combination) from prediction models are implemented to trigger earlier action and potentially, treatment, that are targeted to specific diseases or disease states. Alerts, notifications, and visualizations can be personalized for each patient and their respective patient information. For example, the prediction model can generate visual content to be displayed on user devices. Examples of visual content includes predicted patient outcomes, recommendations for patient treatment, and patient vital sign information. Because outputs are personalized for particular patients, the predictions, visualizations, and recommendations are more accurate and can lead to more efficient adjustments to a patient's treatment regime.

FIG. 4C depicts an exemplary operating environment of the continuous analyte monitoring system in a hospital deployment that includes multiple user devices 420A-C. User devices 420A-C are devices that serve as display interfaces for displaying and/or recording visualizations provided by prediction model 402A. Examples of user device 420A-C include health care provider (HCP) devices, nurse devices, bedside monitors, and EMR dashboards or patient records. HCP devices and nurse devices are practitioner-facing devices within a hospital setting such as mobile devices and workstations. Examples of visualizations 422A-C for display on user devices 420A-C include predicted vital sign trends, predicted patient outcomes, predicted dynamic early warning scores, and decision matrices for facilitating patient treatment modification or discharge decisions.

As one example, integrating a visualization, such as trend information and predicted patient outcomes into graphical user interface of user devices 420A-C, such as a bedside monitor, includes modifying the graphical user interface to display real-time data visualization and decision support insights. The visualizations can include time-series graphs, trend visualization, score visualizations, and threshold visualizations (discussed in more detail below).

In some embodiments, visualizations can be displayed in a “sticky” manner on a graphical user interface of a user device and/or a patient monitoring device, such as a bedside monitor. For example, an HCP can transmit a display request to a monitoring device to persistently display selected patient information, such as analyte level information and the rate of change information, on a display of the monitoring device. The selected information can then be displayed persistently by the monitoring device, along with any additional user input such as HCP notes, until the HCP transmits a request to cancel the display of the additional information.

Another example of a visualization is an interactive graphical visualization for receiving additional user input associated with a patient that is linked to a user device and/or a patient monitoring device. For example, an HCP for the patient can provide additional notes and observations about the patient that can be integrated into the patient medical history, and that can be used by the prediction model as part of generating updated predictions and recommendations for the patient. In some embodiments, the visualizations may be combinable with other. For example, additional information from the interactive graphical visualization and/or time-series graphs, trend visualization, score visualizations, and threshold visualizations for the patient may be displayed in the “sticky” manner described above.

### Hospital Deployment

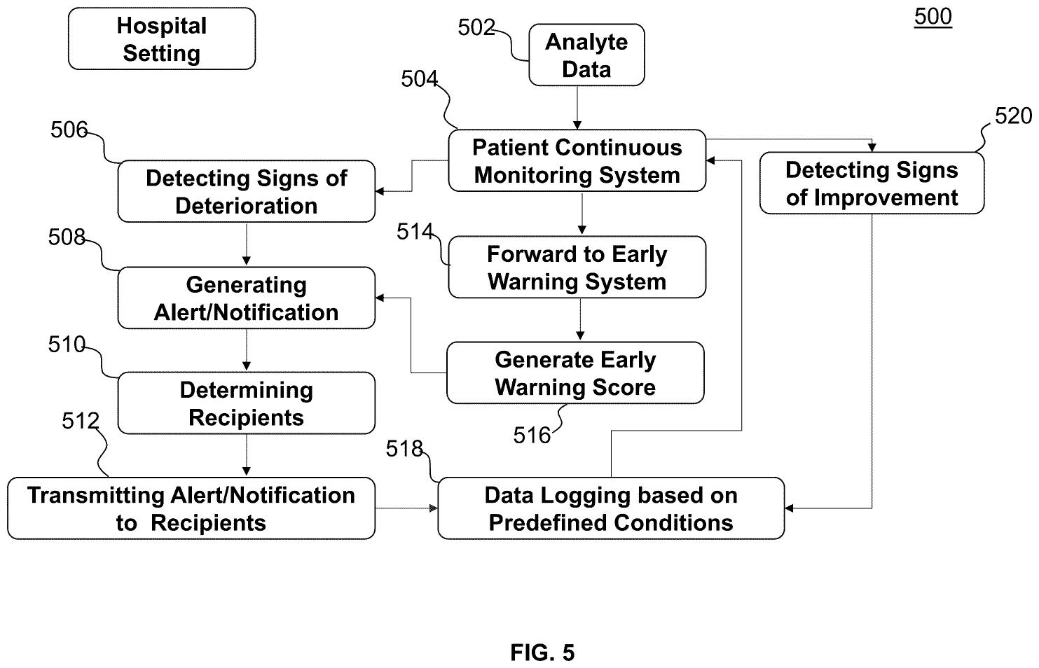

FIG. 5 is a flowchart illustrating a method 500 for utilizing a continuous analyte monitoring system within a hospital environment, according to some embodiments. Method 500 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 5 can be performed by components of CAM system 100, either separately or in a distributed fashion. In such an embodiment, any of these components can execute code in memory to perform certain steps of method 500 of FIG. 5. While method 500 of FIG. 5 will be discussed below as being performed by certain components of CAM system 100, other components can store the code and therefore can execute method 500 by directly executing the code. Accordingly, the following discussion of method 500 will refer to components of FIGS. 1-3 as an exemplary non-limiting embodiment. Moreover, it is to be appreciated that not all steps are needed to perform the disclosure provided herein. Further, some of the functions can be performed simultaneously, in a different order, or by the same components than shown in FIG. 5, as will be understood by a person of ordinary skill in the art.

Hospital admissions generally fall into two broad categories: a) acute care-medical and surgical emergencies and b) planned elective admissions, typically for scheduled treatments or procedures (i.e., planned surgery, interventional procedures, IV infusions requiring hospital monitoring, etc.). CAM system 100 can be utilized during acute illnesses associated with hypoperfusion or circulatory failure, such as heart failure, septic shock, pneumonia with septicemia, and polytrauma to detect levels of lactate as part of generating and improving a prediction model for predicting patient medical outcome and for dynamically recommending treatment adjustments based on monitoring patient response.

In some embodiments, the analyte CAM system 100 is continuously monitoring lactate (i.e. a continuous lactate monitoring system). In some embodiments, CAM system 100 can be utilized to detect the lactate level and rate of change of the lactate level for a patient. CAM system 100 can be utilized to reduce readmission of discharged patients by detecting the lactate level and the rate of change of the lactate level as part of generating and improving a prediction model for predicting patient medical outcome and for dynamically recommending treatment adjustments based on monitoring patient response. In some embodiments, CAM system 100 can dynamically adjust its recommendation to account for patient medical history, patient procedure history, physical exertion/exercise, current vital signs, genomics, comorbidities, body temperature, pulse rate, breathing rate, blood pressure, biometrics, age, trend information, historical data and trends of previous patients, and any combination thereof.

CAM system 100 can be utilized to reduce readmission, within a predetermined period of time after discharge (e.g., 10 days, 20 days, 30 days), for patients suffering from, or admitted for, heart failure or sepsis by detecting the lactate level and the rate of change of the lactate level as part of generating and improving a prediction model for predicting patient medical outcome and for dynamically recommending treatment adjustments based on monitoring patient response. In some embodiments, monitoring patient response can include utilizing data provided by motion sensors attached to the patient to determine and correlate levels of physical activity with patient treatment and response. The predetermined period of time can be configurable and adjusted based on conditions specific to the user such as medical history (disease, comorbidities, prior treatment, analyte trends), sex, age, and other user information.

CAM system 100 can be utilized to reduce readmission of discharged patients by detecting the lactate level and the rate of change of the lactate level, and then using that data to generate a prediction model for predicting which patients should not be discharged, which patients require further medical intervention, which patients require further medical observation, which patients are eligible to be discharged, and to where patients are eligible to be discharged.

CAM system 100 can be utilized to reduce readmission of discharged patients by detecting the lactate level and the rate of change of the lactate level, and then using that data to generate a prediction model for predicting which patients should not be discharged.

CAM system 100 can be utilized to reduce readmission, within 30 days of discharge, for patients suffering from, or admitted for, heart failure or sepsis by detecting the lactate level and the rate of change of the lactate level, and then using that data to generate a prediction model for predicting which patients should not be discharged.

CAM system 100 can be utilized to reduce readmission of discharged patients by detecting the lactate level and the rate of change of the lactate level, and then using that data to generate a prediction model for predicting which patients require further medical intervention. Non-limiting examples of intervention include: fluid resuscitation, intravenous therapy, surgical intervention, administration of electrolytes, administration of antibiotics, administration of O₂, administration of insulin, administration of glucose, and any combination thereof.

CAM system 100 can be utilized to reduce readmission of discharged patients by detecting the lactate level and the rate of change of the lactate level, and then using that data to generate a prediction model for predicting which patients require further medical observation. Examples of medical observation include recommendations for monitoring patent condition and a monitoring plan. In some embodiments, the prediction model predicts which patients require additional medical intervention. Examples of medical intervention include recommendations for actions or treatment, and a treatment plan for the patient.

CAM system 100 can be utilized to reduce readmission of discharged patients by detecting the lactate level and the rate of change of the lactate level, and then using that data to generate a prediction model for predicting which patients are eligible to be discharged and to where patients are eligible to be discharged.

CAM system 100 is configured to continuously monitor lactate levels of any patient in a hospital setting. In some embodiments, analyte sensor 102 of CAM system 100 can be implemented as part of continuous lactate monitor system for measuring lactate levels in dermal interstitial fluid of patients, which allows closer monitoring of the lactate levels of patients in the acute hospital setting. In some embodiments, analyte sensor 102 of CAM system 100 is implemented as part of continuous lactate monitor system for measuring rate of change of the lactate level in dermal interstitial fluid of patients, which allows closer monitoring of the rate of change of the lactate level of patients in the acute hospital setting. CAM system 100 also provides a novel means of measuring lactate levels in a clinical setting while providing an increased level of surveillance of a patient's lactate levels with the potential of reducing the requirement for multiple blood tests, allowing real-time monitoring of the patient's response to treatment, and allowing the early identification of deterioration.

Continuous lactate monitoring can be used to monitor patients by screening for signs of deterioration and response to treatment, thereby allowing for earlier identification of patient decline and earlier intervention/initiation of treatment. Such earlier intervention/initiation of treatment will lead to: a) a reduction in patient mortality; b) a reduction in intensive care admission; c) a reduction in readmission of discharged patients; d) a determination of when medical intervention is required; e) a determination of when medical observation is required; and f) shorten inpatient and ICU length of stay (determination of which patients are eligible to be discharged).

Analyte sensor 102 can be implemented as a biowearable analyte (e.g., lactate, glucose, dual) sensor. Benefits of using a biowearable sensor include but are not limited to being less expensive, non-invasive/non-permanent, no procedure/no in-person visit, no expert team and equipment, easy start-up, easy data capture, easy data sharing with patient and provider. Additionally, a biowearable sensor can be updated with new hardware and/or software to provide longer-term improvements including providing an accelerometer/altimeter for activities of daily living, providing a temperature sensor to detect body temperature, providing heart rate and heart rate variability data capture, extending wear-time anywhere from between 5 days to one month, and adding additional biomarkers including proteins.

In step 502, patient data associated with a patient is received by a prediction model (e.g., prediction model 402). The patient data can include analyte data from one or more analyte sensors such as analyte sensor 102. Patient data can also include information received from monitoring devices such as blood pressure information, heart rate information, respiratory rate, oxygen saturation, and body temperature. Patient data can also include information from laboratory tests such as cardiac markers and inflammatory markers. The analyte data can include any analyte including, but not limited to, lactate, glucose, or creatinine. Any combination of analytes can be utilized depending on the use-case deployment for which the predicted patient outcome is being provided. As one non-limiting example, a patient's creatinine data can be input into the prediction model for predicting a length of stay for a patient after cardiac and/or renal surgery. As an additional non-limiting example, the lactate level and rate of change of the lactate level can be input into the prediction model for predicting which patients should not be discharged, which patients require further medical intervention, which patients require further medical observation, which patients are eligible to be discharged, and patient discharge location (e.g. home or a rehabilitation facility).

In step 504, the patient continuous monitoring system processes the patient data received from one or more devices (e.g., analyte sensor, patient monitoring devices, and laboratory tests) as discussed in step 502. Processing the patient data can include transferring the patient data between components (e.g., between analyte sensor 102 and any combination of data receiving device 120, multi-purpose data receiving device 130, and/or user device 135). CAM system 100 is an exemplary implementation of a patient continuous monitoring system. A component (any combination of data receiving device 120, multi-purpose data receiving device 130, and/or user device 135) in CAM system 100 can also receive other information associated with patient including the patient's medical information and trend information (e.g., blood pressure, heart rate, respiratory rate, lactate level, rate of change of the lactate level, treatment history including current and prior medicine). CAM system 100 provides the analyte and medical information to a prediction model for generating a predicted patient outcome based on the analyte and medical information. In some embodiments, the prediction model can be implemented in any combination of data receiving device 120, multi-purpose data receiving device 130, and/or user device 135, or on a per-deployment basis such as prediction models 402A-D discussed with respect to FIG. 4B. Examples of a predicted patient outcome include, but are not limited to, upward or downward trends in patient condition (i.e., patient deterioration), patient response to potential treatment, length of stay following a procedure, and indicators of a potential disease or condition. For example, the prediction model can be trained to predict the length of a patient's stay following a procedure based on the patient's continuous lactate information. In another example, the prediction model can be trained to predict which patients should not be discharged, which patients require further medical intervention, which patients require further medical observation, which patients are eligible to be discharged, and to properly identify or suggest the most appropriate discharge location (i.e., home, rehab, nursing facility) based on the patient's continuous lactate information. In another example, the prediction model can be trained to provide recommendations for a sequence of decision points, where the sequence of decision points can depend on a patient's disease and medical conditions.

In step 506, output of the prediction model can be used by CAM system 100 to detect signs of deterioration in the patient. For example, a prediction model can be implemented in any combination of data receiving device 120, multi-purpose data receiving device 130, and/or user device 135. Each device can be configured to generate a predicted patient outcome based on received analyte information from analyte sensor 102 associated one or more patients. In some aspects, the device can also receive medical information associated the one or more patients and can be further configured to generate the predicted patient outcome based on both the analyte information and the medical information. In some aspects, detection can be based on comparing the predicted patient outcome to predefined threshold values and signs of deterioration can be determined based on the predicted patient outcome exceeding one or more predefined threshold values. In some aspects, the predicted patient outcome can be implemented as any combination of a number value (e.g., between one and a hundred), a color scheme (e.g., green is no action; yellow is monitoring required; red is intervention required), a text message (e.g., “suspected sepsis” or “patient vitals deteriorating”), a visual display such as a trend graph or a decision matrix (e.g., showing patient vitals including the continuously monitored analyte information). In some embodiments, the predicted patient outcome can be included in an electronic medical record (EMR) such as an EMR communication channel or an EMR-defined alert indicator (e.g., a colored icon such as a red exclamation mark to signal an abnormal flow).

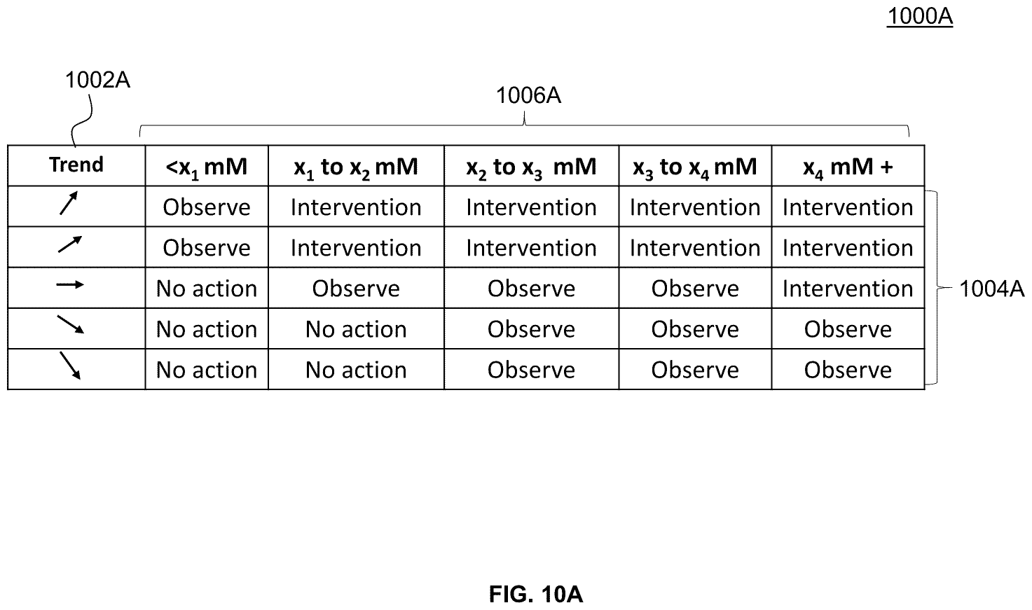

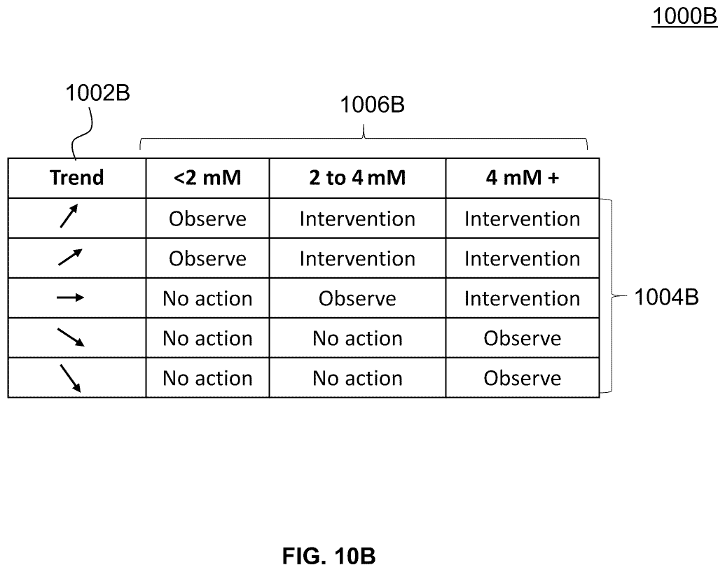

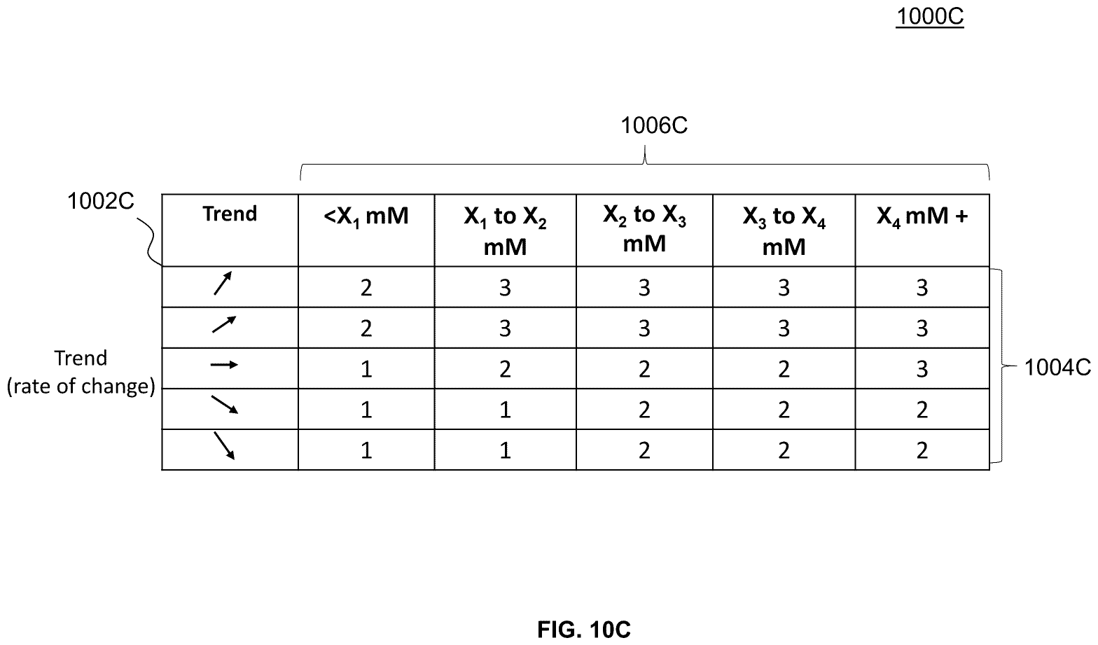

FIGS. 10A-C depict exemplary decision matrices. In some aspects, as exemplified in FIGS. 10A-C, detection can be based on evaluating the patient's lactate levels and the rate of change of the patient's lactate levels against predefined threshold lactate level values and predefined threshold lactate level rates of change to generate a predicted patient outcome. For example, signs of deterioration can be determined by analyzing both the patient's lactate levels and the rate at which those lactate levels are changing over time. Specifically, prediction model 402 can evaluate whether the lactate level and its rate of change exceed or fall below one or more predefined threshold lactate level values and predefined threshold lactate level rates of change that indicate potential patient deterioration. A correlation between these factors can be established by determining how lactate concentration and its rate of change interact-such as, for example, whether an already high lactate level combined with a rapid increase signals a worsening condition. If this combined effect meets certain criteria, such as exceeding a critical threshold, prediction model 402 generates a predicted patient outcome, indicating that an intervention may be needed, and in some embodiments, providing suggestions as to the type and duration of such interventions. In some embodiments, decision matrices of FIGS. 10A-C depict lactate measurements. In some embodiments, other analytes, either singularly or in combination with lactate, can be utilized as part of the decision matrices for determining signs of deterioration and as the basis of determining predetermined recommendations for patient action.

In some embodiments, prediction model 402 can be configured to update decision matrices, including trend visualizations, score visualizations, and threshold visualizations. For example, trend visualizations can include one or more metrics associated with continuously monitored analyte information. Examples of such metrics include rate of change, trend, duration of time within one or more threshold ranges, area under the curve, and percentage change. The combination of metrics selected as part of trend visualizations can be based on one or more of patient medical history, disease state, patient condition, and health care provider (HCP) input. Prediction model 402 can be configured to determine metrics that are most relevant to the patient being monitored based on this information, and can be further configured to dynamically update the visualization as additional patient data, HCP input, and other information becomes available.

Score visualizations and threshold visualizations can also be dynamic and personalized based on various factors, such as patient information, disease, current analyte information, and HCP input and based on patient's current condition. For example, different diseases can be linked to different analytes being monitored (e.g., any combination of glucose, ketone, and lactate) and different threshold visualizations for the respective analytes. Threshold visualizations can be further configured based on the particular analyte (or analytes) that are being continuously monitored for the patient.

Score visualization of decision matrices can be configured to depict any combination of dynamic early warning scores or recommended actions (e.g., “no action,” “observe,” or “intervention”). Dynamic early warning scores can be personalized for particular diseases or disease states. As a non-limiting example involving continuously monitoring lactate values, the dynamic early warning score can be associated with a lactate variability index that numerically represents variability of lactate values between a first and second threshold (e.g., a low threshold and a high threshold). A lactate variability index can be a measure of the variation in lactate levels within a patient that represent fluctuations between particular thresholds (e.g., low and high threshold) over a given period. The lactate variability index can quantify how dynamic are the lactate levels, which can provide insight into patient condition. In some embodiments, the index can be represented as a time-series graph, depicting lactate levels over time, with color-coded ranges depicting various threshold ranges (e.g., normal, moderate, critical states). Another embodiment for lactate variability index includes a heatmap which can depict variances in lactate values using different colors to represent the degree of fluctuation over time.

When implemented as part of a dynamic early warning score, a lactate variability index can be implemented as a single value summarizing lactate variance on a scale (e.g., 0=stable and 5=very unstable) or as a trend-based numerical indicator such as the lactate variance over a period of time which can reflect how quickly lactate is changing (e.g., >2 mM/L/hour can indicate worsening condition).

Threshold visualization of decision matrices can be associated with thresholds for one or more metrics of continuously monitored analyte information. Examples of metrics include trend information, percentage change, area under the curve, and duration of time below, within, or above threshold ranges.

For example, decision matrix 1000A of FIG. 10A can include trend visualization 1002A, score visualization 1004A, threshold visualization 1006A. In some embodiments, trend visualization 1002A can reflect varying trend rates (e.g., rapid rise, rise, no change, decline, rapid decline), score visualization 1004A can reflect recommended action items to be performed by CAM system 100, and threshold visualization 1006A can reflect analyte values that, in combination with trend rates from trend visualization 1002A, can be used as inputs for detecting the appropriate action (or that no action is necessary).

In some embodiments, decision matrix 1000A can be generated on a patient specific basis such that each decision matrix 1000A can be associated with a unique patient identifier. In some embodiments, decision matrix 1000A can be dynamically updated as needed by a machine learning model (such as prediction model 402) based on patient specific data (such as past medical history, current medical history, inputs from an electronic health record) and user input (such as inputs received from a doctor's user device). Score visualization 1004A of decision matrix 1000A in FIG. 10 indicates potential notifications or actions based on trend data in trend visualization 1002A and analyte data in threshold visualization 1006A. Examples of notifications include “no action” (e.g., the patient's information indicates normal conditions), “observe” (e.g., the patient's information indicates potential issues) and “intervention” (e.g., sending notifications or alerts to determined recipients, including predetermined actions such as fluid resuscitation, antibiotics, or oxygen, just to name a few examples).

Examples of dynamically updating a decision matrix include adjusting display of trend visualization 1002A, score visualization 1004A, threshold visualization 1006A as new data is continuously collected and provided to the machine learning model. For example, a patient in a hospital setting may be continuously monitored via one or more patient monitors that continuously provide new data to the machine learning model. Examples of data from these patient monitors include patient vital signs, patient analyte data, and HCP notes or observations.

Trend information (e.g., from trend visualization 1002A of decision matrix 1000A in FIG. 10A) refers to trends in the patient's analyte and medical history over a certain period of time (e.g., 8 hours, 12 hours). A dynamic early warning score differs from a conventional early warning score because it can be continuously updated based on continuously monitored analyte data from the patient as well as the patient's medical information.

Based on the dynamic early warning score (e.g., score visualization 1004A of decision matrix 1000A in FIG. 10A), CAM system 100 can generate an alert and/or notification in 508. For example, if the dynamic early warning score is greater than predefined thresholds, CAM system 100 can be configured to generate one or more alerts and/or notifications to different caregivers in the hospital (e.g., as indicated by recommended actions in score visualization 1004A from decision matrix 1000A).

Decision matrices can be customized for specific diseases. For example, FIG. 10B depicts decision matrix 1000B with trend visualization 1002B, score visualization 1004B, threshold visualization 1006B, with analyte values (e.g., lactate) customized for a particular condition (e.g., heart disease). Decision matrix 1000B may also be dynamically updated as discussed above with respect to decision matrix 1000A Different conditions can have different values and/or metrics for trend visualization 1002B, score visualization 1004B, threshold visualization 1006B. In this particular embodiment, threshold visualization 1006B depicts a first threshold of less than 2 mM, 2 to 4 mM, and above 4 mM. Score visualization 1004B is represented here as a recommendation which can correspond to a particular score or metric associated with values in trend visualization 1002B and threshold visualization 1006B. For example, recommendations can include “no action,” “observe,” and “intervention.”

In some embodiments, decision matrix 1000B may represent a particular parameter associated with an analyte level, such as “time above threshold,” and/or “coefficient of lactate variability,” and the decision matrix 1000B may be transmitted to an HCP device for visualization presentation. As one example, the “time above threshold” parameter may comprise a period of time above 4 mmol/L for lactate over a 24 hour period and may be compared to a historical “time above threshold” parameter (e.g., prior 24 hour period), to a baseline set by a clinician, or to an accepted reference value or target range for that parameter. As another example, the “coefficient of lactate variability” (CV) parameter may indicate the relative variability of lactate measurements over a 24 hour period, calculated as the ratio of standard deviation to the mean, where a higher CV indicates greater lactate variability, while a lower CV indicates less lactate variability.

FIG. 10C depicts decision matrix 1000C. The predicted patient outcome can be a number value associated with a proposed predetermined course of action. Predetermined actions can be configurable based on any number of different parameters including the disease, the patient cohort, patient medical history, hospital settings, home settings, just to name a few examples. Decision matrix 1000C may also be dynamically updated as discussed above with respect to decision matrix 1000A.

As a non-limiting example, FIG. 10C depicts trend visualization 1002C, threshold visualization 1006C, and score visualization 1004C. Score visualization 1004 can include predetermined values corresponding to a warning score associated with a patient condition and potential recommendation. For example, a predetermined value (e.g., “3”) can be associated with one or more particular analyte levels and rates of change can be correlated to a predetermined course of patient action (e.g., intervention required). Another predetermined value (e.g., “2”) which is associated one or more different analyte levels and rates of change can be correlated to another predetermined course of action (e.g., observation required). And yet another predetermined value (e.g., “1”) which is associated with one or more different analyte levels and rates of change can be correlated to another predetermined course of action (e.g., no action required). It is understood that the disclosure is not limited to these values and courses of action and that other indicators beside number value can be used and other proposed courses of actions can be used.

Non-limiting examples of other indicators include: a text message or a voice memo (e.g., “rate of change of patient lactate level increasing/decreasing”; “patient lactate level increasing/decreasing”; “no change to patient lactate level/rate of change of patient lactate level”; etc.), a visual display such as a trend graph or a table (e.g., showing patient vitals including the continuously monitored lactate information; showing a graph of change in lactate level over time; showing a graph of change in rate of change of lactate level over time; etc.).

Non-limiting examples of other proposed courses of action include: designations of specific types of intervention (e.g., fluid resuscitation; intravenous therapy; surgical intervention; administration of electrolytes; administration of antibiotics; administration of O₂; administration of insulin; administration of glucose; etc.), designations of specific types of observation (e.g., self-observation; nurse observation; doctor observation; temporary observation; continuous observation; qualitative discussion of condition with patient (e.g. “How do you feel?”); computer assisted observation; etc.); discharge from hospital; transfer to more-intensive care; transfer out of more-intensive care; review patient medical history; confer with other medical staff to determine course of action; contact specialists.

In some aspects, the arrangement of the number values associated with a proposed course of action shown in FIG. 10C is adjusted (i.e. a 2 may become a 1 and vice-versa; a 2 may become a 3 and vice-versa; 3 may become 1 and vice-versa; etc.). In some aspects, the arrangement of the number values associated with a proposed course of action, and the predicted patient outcome, shown in FIG. 10C are adjusted to account for patient medical history, patient procedure history, physical exertion/exercise, current vital signs, genomics, comorbidities, body temperature, pulse rate, breathing rate, blood pressure, biometrics, age, trend information, historical data and trends of previous patients, and any combination thereof. These factors may be weighted based on a patient's current condition such that some factors may be given more weight as part of predicting the patient outcome. For example, for a patient in the hospital or after a high-risk surgery may have different weighted factors for a patient at home with potential heart failure conditions.

In some aspects, the number of predefined threshold lactate level values indicated on FIG. 10C is adjusted (i.e. an additional range “from X₄ to X₅ mM” can be added; an additional range “above X₅ mM” can be added; the range “above X₄ mM” can be removed making “above X₃ mM” the new highest range; etc.). In some aspects, the number of predefined threshold lactate level rates of change indicated on FIG. 10C is adjusted (i.e. an additional rate of change row is added between, above, or below existing rows; a rate of change row can be deleted out of the table; etc.).

In some aspects, all possible correlations of the lactate level value and the lactate level rate of change will generate a number value associated with a proposed course of action and predicted patient outcome (i.e. FIG. 10C adjusted to have infinite rows and columns, displaying all possible lactate level rates of change and lactate level values respectively).

In step 508, a component in CAM system 100 (e.g., any combination of data receiving device 120, multi-purpose data receiving device 130, and/or user device 135) can generate an alert and/or notification based on the detection of any signs of deterioration in the patient. In 508, a component in CAM system 100 (e.g., any combination of data receiving device 120, multi-purpose data receiving device 130, and/or user device 135) can generate an alert and/or notification based on the detection of increased or decreased lactate levels and positive or negative rate of change of lactate levels in a patient. Alerts and notifications include the predicted patient outcome. An alert differs from a notification with regard to any required action that can be required based on the predicted patient outcome. For example, an alert can require an immediate action or treatment to be administered (i.e. fluid resuscitation, intravenous therapy, surgical intervention, administration of electrolytes, administration of antibiotics, administration of O₂, administration of insulin, administration of glucose), either manually by a caregiver or automatically, by a medical device that is already connected to the patient. The alert can include one or more treatment recommendations for addressing the predicted patient outcome, including observation and medical intervention. In contrast, a notification can simply provide the predicted patient outcome and does not require any immediate action or treatment. Accordingly, alerts and notifications can be used based on a severity of the predicted patient outcome. CAM system 100 can generate the treatment recommendations based on the predicted patient outcome generated in 506. An alert and/or notification can be generated to notify one or more recipients of the patient's predicted outcome. An alert and/or notification can be documented in an electronic medical record (EMR). An alert and/or notification can be sent through in-EMR communication channels or as an EMR-defined abnormal value indicator (i.e., red exclamation mark to indicate abnormal conditions). An alert and/or notification can be sent through a Best Practice Advisory system or a Clinical Decision Support system. In some embodiments, an alert and/or notification can further be sent to a 3^{rd} party clinical monitoring service (e.g., a telehealth company, a remote monitoring company, a telemonitoring company).

In step 510, a component in CAM system 100 (e.g., any combination of data receiving device 120, multi-purpose data receiving device 130, and/or user device 135) can determine the recipients for receiving the generated alert and/or notification. Examples of recipients include health care providers such as nurses and doctors, as well as designated caregivers. Another example of a recipient is an intermediate alert verification system, such as a platform within a machine learning model trained to determine if an alert is accurate and/or gather additional data, then, if warranted, pass their determination and data onto the patient and/or healthcare personnel (RPM model). The intermediate alert verification system can be implemented between the patient and the hospital system. The recipients can be predetermined based on different predicted patient outcomes. For example, if the predicted patient outcome identifies a particular disease (e.g., heart failure, sepsis), CAM system 100 can identify a predetermined recipient based on the identified disease. As another example, if the predicted patient outcome identifies a downward trend in the patient's condition, CAM system 100 can identify the predetermined recipient based on the severity of the downward trend (e.g., a first threshold level can trigger an alert and/or notification to a nurse and a second threshold level can trigger an alert and/or notification to the doctor).

In step 512, the alert and/or notification is transmitted to a device associated with the predetermined recipient. Examples of devices associated with include their personal devices (with dedicated software installed) for receiving the alert and/or notification.

In step 514, the generated predicted patient outcome can be forwarded to an early warning system. Some hospitals use an early warning system to screen patients for evidence of deterioration or signs of improvement by calculating an early warning score. Early warning scores are typically based on the patient's vital signs such as heart rate, blood pressure, body temperature and respiratory rate. CAM system 100 improves the accuracy of predicting deterioration in patients and transmission of the alerts by combining the patient's analyte information with an early warning score system.

In step 516, CAM system 100 generates a dynamic early warning score based on any combination of the patient's analyte information (provided by a continuous analyte monitoring sensor) and the patient's medical information, including vital signs and trend information. Trend information refers to trends in the patient's analyte and medical history over a certain period of time (e.g., 8 hours, 12 hours). A dynamic early warning score differs from a conventional early warning score because it can be continuously updated based on continuously monitored analyte data from the patient as well as the patient's medical information.

Based on the dynamic early warning score, CAM system 100 can generate an alert and/or notification in 508. For example, if the dynamic early warning score is greater than predefined thresholds, CAM system 100 can be configured to generate one or more alerts and/or notifications to different caregivers in the hospital.

In step 518, remote application server 155 can receive and store the patient information in a remote database, such as in the patient's electronic medical records (EMR). The patient information can be transmitted (e.g., by any combination of data receiving device 120, multi-purpose data receiving device 130, and/or user device 135) to remote application server based on any combination of predefined conditions, such as a schedule (e.g., daily, weekly), thresholds (e.g., if a dynamic early warning score exceeds an alert threshold) and the predicted patient outcome (e.g., if the predicted outcome exceeds a severity level).

In step 520, output of the prediction model can be used by CAM system 100 to detect signs of improvement in the patient. In some aspects, detection can be based on comparing the predicted patient outcome to predefined threshold values and signs of improvement can be determined based on the predicted patient outcome exceeding one or more predefined threshold values. In some aspects, detection can be based on comparing the patient lactate level and patient rate of change of lactate level to predefined threshold lactate level values and predefined threshold lactate level rates of change. Signs of improvement can be determined based on the patient lactate level and patient rate of change of lactate level exceeding or being less than one or more predefined threshold lactate level values and predefined threshold lactate level rates of change.

In some embodiments, CAM system 100 distinguishes between potential disease conditions (e.g., a potential heart failure decompensation event) and conventional physical activity using patient information (e.g., vitals) from machines connected to the patient such as a heart rate monitor, blood pressure monitor, temperature monitor, skin surface temperature monitor, and activity monitor (e.g., accelerometer, step counter). Based including additional patient information as part of predicting patient condition, CAM system 100 can reduce frequency of false alarms or alerts and improves accuracy of predicted patient conditions.

CAM system 100 can be configured to provide vital sign information in addition to continuously monitored analyte information for generating output in step 520. As an example, the prediction model can be configured to determine a link between rate of change of continuously monitored analyte information and activity information. The prediction model can be configured to determine that changes in analyte information can be based on activity information (e.g., a spike in lactate value can be due to physical activity) and further configured to identify differences between activity-based elevation vs. deterioration elevation based on pattern recognition.

In some embodiments, an output of the prediction model includes a combination of a graphical visualization (e.g., a color-coded dynamic early warning score, a trend graph, an alert or notification to be transmitted to a recipient device, a visualization such as predicted trend or score for display on a component of CAM system 100, such as a user device or a bedside monitor associated with the patient). For example, CAM system 100 can be configured to generate different visualization components based on the output of the prediction model, where the different visualization components can be customized based on the patient condition, disease, and/or HCP preferences. For example, output of the prediction model may be based on a widely used, medically accepted heart failure classification system, such as the New York Heart Association (NYHA) functional classification that categories patients based on their physical activity limitation, ranging from Class I where a patient has no symptoms nor limitation in physical activity, to Class IV where a patient is unable to carry on any physical activity without discomfort. In this embodiment, the output of the prediction model can indicate that a patient is at risk of transitioning from a Class II to Class III functional class, or from slight limitation in physical activity to marked limitation in physical activity, and CAM system 100 can be configured to generate one or more graphical visualizations based on that prediction, such as generating a trend visualization for the bedside monitor, an alert or notification to the nurses station or HCP device, and a predicted trend graph or table for display on a designated display within the hospital.

CAM system 100 can be configured to generate different combinations of these graphical visualizations based on different predictions, different diseases, different patients, different HCPs, and different patient disease progressions. Accordingly, the graphical visualizations can be personalized for specific patients and conditions to increase the accuracy of the prediction and likelihood that the visualizations will be helpful as part of the decision support system.

As another example, prediction model can be configured to use data from any one of the patient machines as part of performing a sanity check on predicted patient conditions, such as whether a rise in analyte values is because of patient condition or patient activity. For example, CAM system 100 can retrieved temperature information, such as from a skin surface temperature sensor for the relevant time period and cross-reference the temperature information with the continuous analyte data and other vital signs. The prediction model can be configured to identify a rise in analyte levels as being activity related based on a certain pattern of vitals which are linked to patient activity, such as higher temperature, rise in lactate levels, and activity information (accelerometer data and/or number of steps during the relevant period).

### Home Deployment

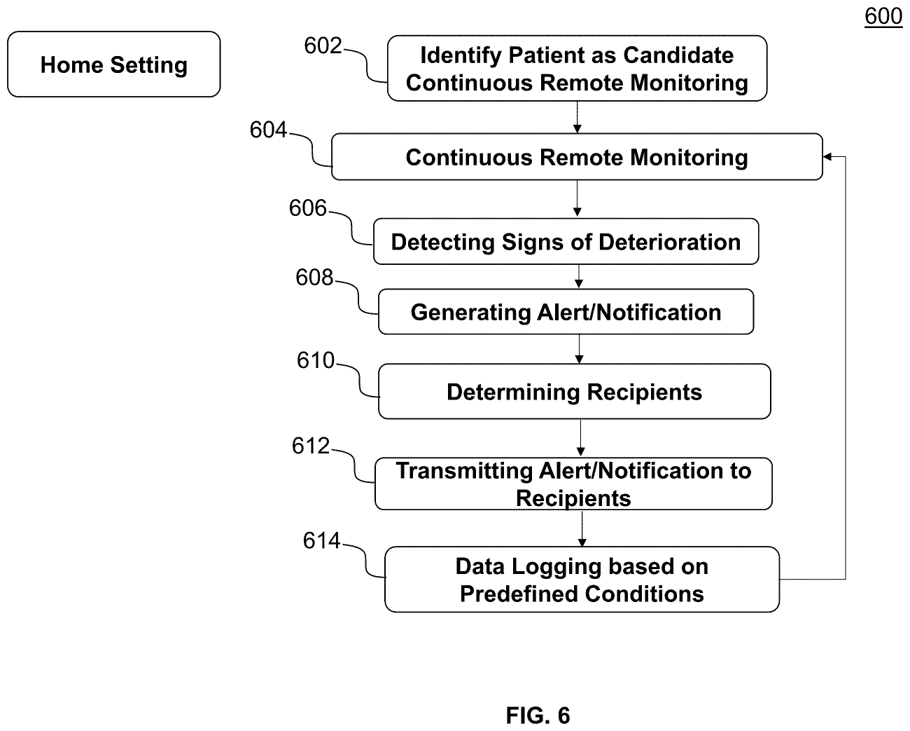

FIG. 6 is a flowchart illustrating a method 600 for utilizing a continuous analyte monitoring system within a home environment, according to some embodiments. Method 600 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 6 can be performed by a components of CAM system 100, either separately or in a distributed fashion. In such an embodiment, any of these components can execute code in memory to perform certain steps of method 600 of FIG. 6. While method 600 of FIG. 6 will be discussed below as being performed by certain components of CAM system 100, other components can store the code and therefore can execute method 600 by directly executing the code. Accordingly, the following discussion of method 600 will refer to components of FIGS. 1-3 as an exemplary non-limiting embodiment. Moreover, it is to be appreciated that not all steps may be needed to perform the disclosure provided herein. Further, some of the functions can be performed simultaneously, in a different order, or by the same components than shown in FIG. 6, as will be understood by a person of ordinary skill in the art.

Continuous lactate monitoring by CAM system 100 can be used to monitor patients in the home setting for signs of deterioration, thereby allowing earlier identification and proactive treatment to prevent hospitalization and deterioration. Measurement of lactate levels can be provided to a prediction model for providing an increased level of surveillance of a patient's lactate levels while the patient is at home. CAM system 100 can be configured to coordinate analyte information to a healthcare practitioner for early identification of deterioration and earlier intervention even when the patient is outside of the hospital setting.

Following discharge from hospital, or in patients identified at risk of deterioration in the home or long-term care setting, continuous analyte monitoring by CAM system 100 could be used to screen for signs of deterioration. Under the remote supervision of a healthcare professional, this could allow the earlier detection of patient decline leading to earlier intervention, carrying the potential to reduce patient mortality.

CAM system 100 can be utilized for earlier identification of patients requiring readmission to the hospital by detecting the lactate level and the rate of change of the lactate level, and then using that data to predict which patients should be readmitted to the hospital. CAM system 100 is capable of earlier identification of patients requiring readmission then conventional methods, which allows for the earlier detection of patient decline leading to earlier intervention, carrying the potential to reduce patient mortality. In some embodiments, CAM system 100 can dynamically adjust its prediction to account for patient medical history, patient procedure history, physical exertion/exercise, current vital signs, genomics, comorbidities, body temperature, pulse rate, breathing rate, blood pressure, biometrics, age, trend information, historical data and trends of previous patients, and any combination thereof.

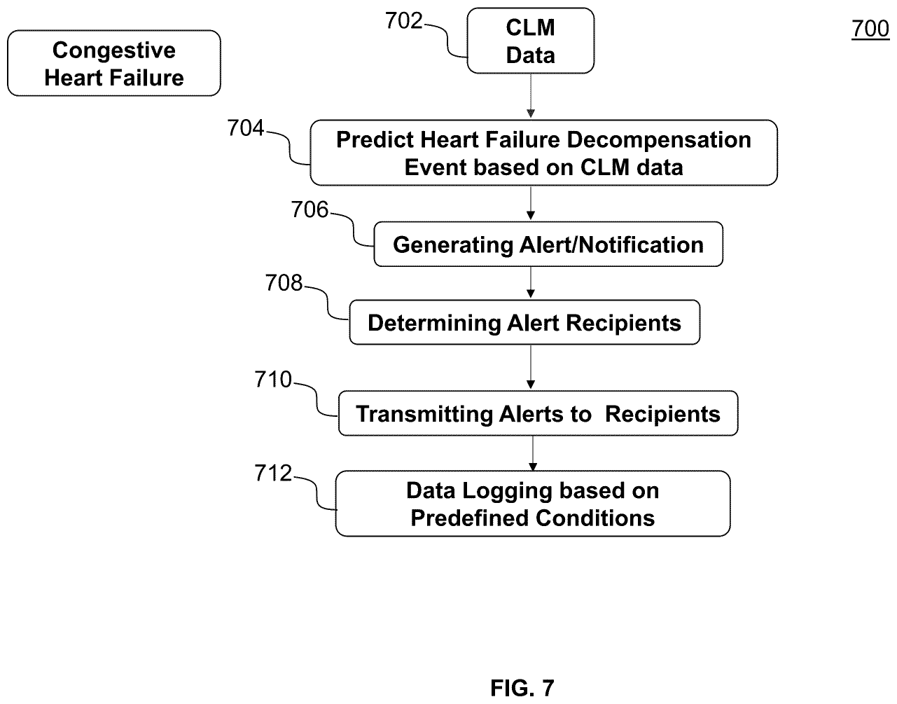

In some embodiments, trend and analyte data of patients prematurely discharged then readmitted to a hospital is used to better inform future predictions of whom is eligible to be discharged or, if already discharged, at higher risk of readmission, thus reducing patient readmission. Trend and analyte data of patients prematurely discharged then readmitted to a hospital is an aspect of “historical data and trends of previous patients” that can be used when adjusting the number value associated with a proposed course of action as described in FIG. 7.

CAM system 100 can be utilized to reduce patient readmission by detecting the lactate level and the rate of change of the lactate level, and then using that data to propose an appropriate course of action not necessitating readmission to the hospital (i.e. self-monitoring, in-home nurse/aid intervention, sending a notification of the patient's status to their doctor).

CAM system 100 can be utilized to reduce readmission, within 30 days of discharge, for patients suffering from, or admitted for, a lactate related condition or a chronic disease state in which lactate may rise (i.e., heart failure; sepsis; pulmonary, circulatory, neurological, hepatic, and/or renal systems disorders) by detecting the lactate level and the rate of change of the lactate level, and then using that data to propose an appropriate course of action not necessitating readmission to the hospital (i.e. self-monitoring, in-home nurse/aid intervention, sending a notification of the patient's status to their doctor).

In step 602, CAM system 100 receives patient identification of any patients that are identified candidates for continuous remote monitoring outside of the hospital. For example, during discharge from a hospital or during a visit to the doctor's visit, an HCP can determine that the patient is at risk for certain conditions that would merit continuous remote monitoring including the continuous remote monitoring of a patient's analyte levels. Once identified, a patient identifier associated with the patient can be transmitted to CAM system 100 to indicate that the analyte sensor 102 associated with the patient is authorized to communicate remotely with components of the CAM system 100.

Accordingly, in some embodiments, the recommendation generated by CAM system 100 can include additional instructions to be performed by the patient after discharge. These additional instructions can include a follow-up plan with steps such wearing a continuous analyte sensor for a predetermined period of time (e.g., 1 week, 2 weeks) after discharge, activity recommendations, diet recommendations, and alerts/notifications for following up with the HCP.

In step 604, a component of CAM system 100 processes analyte data from the patient. For example, analyte sensor 102 associated with the patient can provide analyte information to any combination of data receiving device 120, multi-purpose data receiving device 130, and/or user device 135. In the home setting, analyte sensor 102 can be physically located in a different location and/or building than one or more components of CAM system 100. In some aspects, analyte sensor 102 is configured to communicate with multi-purpose data receiving device 130 which is then further configured to relay the analyte information from analyte sensor to one or more other components in CAM system 100 over a network, such as a Wi-Fi network or a cellular network. Examples of a multi-purpose data receiving device 130 in this embodiment include personal devices (e.g., mobile phone, tablet) associated with the patient with dedicated software for communicating with the analyte sensor 102. Accordingly, analyte sensor 102 can communicate analyte information to devices associated with designated recipients who can be located remotely from the patient. CAM system 100 can also process other information associated with patient including the patient's medical information and trend information (e.g., blood pressure, heart rate, respiratory rate, treatment history including current and prior medicine).

CAM system 100 provides the analyte and medical information to a prediction model for generating a predicted patient outcome based on the analyte and medical information. In some embodiments, prediction model can be implemented in any combination of data receiving device 120, multi-purpose data receiving device 130, and/or user device 135, which can be configured to generate a predicted patient outcome based on the analyte data, and in some additional embodiments, other medical information (e.g., patient's vitals). Additional medical information includes previous hospital visits, medical conditions prior to discharge, reoccurrence of previous conditions such as sepsis, heart failure, decompensation events, and historical vital information. CAM system 100 can also be configured to access emergency medical record systems (EMR) to retrieve the additional medical information.

In step 606, output of the prediction model can be used by CAM system 100 to detect signs of deterioration in the patient. In some aspects, detection can be based on comparing the predicted patient outcome to predefined threshold values and signs of deterioration can be determined based on the predicted patient outcome exceeding one or more predefined threshold values. In some embodiments, an output of the prediction model includes a combination of a graphical visualization (e.g., a color-coded dynamic early warning score, a trend graph, an alert or notification to be transmitted to a recipient device, a visualization such as predicted trend or score for display on a component of CAM system 100, such as a user device).

In step 608, a component of CAM system 100 (e.g., any combination of data receiving device 120, multi-purpose data receiving device 130, and/or user device 135) can generate an alert and/or notification based on the detection of any signs of deterioration in the patient. Alerts and notifications include the predicted patient outcome. The alert can include one or more treatment recommendations for addressing the predicted patient outcome. A component of CAM system 100 can generate the treatment recommendations based on the predicted patient outcome generated in 506 and include the generated recommendations in the alert. Accordingly, an alert and/or notification can be generated to notify one or more recipients of the patient's predicted outcome.

In some embodiments of this deployment, CAM system 100 can be configured to utilize patient location information (e.g., when the patient is not located in the hospital) and outputs can be configured based on patient location. For example, a severe alert can dictate recipient devices (e.g., 911) and actions, such as calling a hospital or emergency services. Potential recipients can include caregivers, PCP, patient, and remote monitoring services based on severity of the alert/notification. CAM system 100 can also be configured to ping or otherwise attempt to contact a patient prior to elevating alert conditions.

In step 610, a component in CAM system 100 (e.g., any combination of data receiving device 120, multi-purpose data receiving device 130, and/or user device 135) can determine the recipients for receiving the generated alert and/or notification. The recipients can be predetermined based on different predicted patient outcomes, proximity to the patient, time of day, and level of severity of the predicted outcome. When implemented in a home setting, the intended recipient can be identified based on their capability to more quickly respond to any alerts that require immediate action.

In step 612, the alert and/or notification is transmitted to a device associated with the predetermined recipient. Examples of devices associated with the predetermined recipient include their personal devices (with dedicated software installed) for receiving the alert and/or notification.

In step 614, remote application server 155 can receive and store the patient information including the predicted patient outcomes, the signs of deterioration, any recommendations, and any other information considered as part of predicted patient outcome in a remote database, such as in the patient's electronic medical records (EMR) as described above in step 518.

### Heart Failure Deployment

FIG. 7 is a flowchart illustrating a method 700 for utilizing a continuous analyte monitoring system in the detection of heart failure conditions, according to some embodiments. Method 700 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 7 can be performed by a components of CAM system 100, either separately or in a distributed fashion. In such an embodiment, any of these components can execute code in memory to perform certain steps of method 700 of FIG. 7. While method 700 of FIG. 7 will be discussed below as being performed by certain components of CAM system 100, other components can store the code and therefore can execute method 700 by directly executing the code. Accordingly, the following discussion of method 700 will refer to components of FIGS. 1-3 as an exemplary non-limiting embodiment. Moreover, it is to be appreciated that not all steps can be needed to perform the disclosure provided herein. Further, some of the functions can be performed simultaneously, in a different order, or by the same components than shown in FIG. 7, as will be understood by a person of ordinary skill in the art.

Heart failure (HF) is a condition where the cardiac output is inadequate to meet the demands of the body. HF presents with symptoms of shortness of breath, particularly when lying flat, swelling of the legs and ankles, and reduced ability to exercise. Patients with heart failure vary in severity from those who develop symptoms on exertion whereas the more severe patients have symptoms at rest. HF is often a chronic condition, where the patient experiences a slow, decline in cardiac function. However, some patients can experience a sudden deterioration in cardiac function, known as acute heart failure, and frequently requires adjustment of treatment and often needing hospitalization. During episodes of heart failure, the heart pumps inadequately and cannot pump blood out to the body normally. Blood backs up in the lungs and increases blood pressure there, referred to as pulmonary hypertension. Inability of the heart to relax appropriately can also cause blood to back up into the lungs, which contributes to pulmonary hypertension. When the heart is not able to pump efficiently, blood can back up into the veins that take blood through the lungs. As the pressure in these blood vessels increases, fluid is pushed into the air spaces (alveoli) in the lungs.

Typically, an implantable pulmonary artery pressure sensor, such as the CardioMEMS HF system is utilized to monitor rising pulmonary artery pressure, which indicates right ventricular congestion (afterload). Monitoring lactate levels through an on-body sensor is non-invasive and can provide information for predicting changes to pulmonary artery pressure. This is possible due to the CAM system 100 utilizing rising lactate as an indicator for a potential heart failure decompensation event.

Accordingly, a prediction model in CAM system 100 can rely on continuous lactate monitoring to monitor blood lactate levels for the purpose of detecting and predicting cardiac events in patients. The prediction model can utilize changes in lactate levels to provide an early and non-invasive means for predicting rising pulmonary artery pressure which can lead to earlier treatment and monitoring. For example, pulmonary artery predictions from the prediction model can prompt adjustment to treatment by one or more clinicians. As previously discussed, the prediction model can be trained using outcome (e.g., health records, patient data, cohort data, prior predictions, etc.) and lactate levels. When trained, current lactate information (e.g., provided by analyte sensor 102) can be input to the prediction model for generating a predicted patient outcome. In some embodiments, other inputs can be provided to the prediction model as well including, but not limited to, patient data and patient vitals.

In HF, the inefficient pumping of the heart leads to a build-up of pressure within the heart. This build-up of pressure is typically measured in the pulmonary artery. The prediction model of CAM system 100 receives lactate information in a patient and can be configured to generate predictions related to a heart failure based on correlating the lactate information with a rise (or decline) in pulmonary pressure. That is, the prediction model can generate a prediction of increased HF risk based on an increase in the lactate levels of the patient.

In some embodiments, in order to distinguish between lactate level increases due to exercise and lactate level increases due to potential heart failure decompensation events, analyte sensor 102 can be configured to include or communicate with an accelerometer and/or altimeter so as to monitor patient activity during a lactate excursion. Patient activity can be used as a factor for predicting heart failure decompensation events, for example, by minimizing significance of lactate level increase during increased levels of patient activity (e.g., which could indicate exercise or physical activity as causing the increase in lactate levels).

In some aspects, the prediction model utilizes cumulative sum lactate to form its prediction for heart failure decompensation events. Cumulative sum lactate is a running total of lactate over a predetermined period of time (e.g., 8 hours, 24 hours, 2 days) and can be used to illustrate the total amount of lactate as it rises over that predetermined period of time.

In step 702, a prediction model of CAM system 100 receives continuously monitored lactate information from one or more patients (e.g., via analyte sensor 102 connected to each respective patient). As previously noted, the prediction model can be implemented within one or more components in CAM system 100, such as any combination of data receiving device 120, multi-purpose data receiving device 130, and/or user device 135.

In step 704, the prediction model can then utilize the CLM data for predicting potential heart failure decompensation events for the patient. The CLM data can be provided in the form of a cumulative sum of lactate levels over a predetermined period of time. This predetermined period of time can be based on any number of factors include HCP preference, desired level of accuracy for the prediction, and determined severity of patient's condition.

In step 706, a component of CAM system 100 (e.g., any combination of data receiving device 120, multi-purpose data receiving device 130, and/or user device 135) can generate an alert and/or notification based on any predicted heart failure decompensation events in the patient. Alerts and notifications can include the predicted patient heart failure decompensation event. The alert can further include one or more treatment recommendations for addressing the predicted patient heart failure decompensation event. A component of CAM system 100 can generate the treatment recommendations based on predicted patient heart failure decompensation event and include the generated recommendations in the alert. Accordingly, an alert and/or notification can be generated to notify one or more recipients of the patient's predicted patient heart failure decompensation event, and an alert can further provide recommendation actions for addressing the predicted event.

In step 708, a component in CAM system 100 (e.g., any combination of data receiving device 120, multi-purpose data receiving device 130, and/or user device 135) can determine the recipients for receiving the generated alert and/or notification. The recipients can be predetermined based on different predicted patient heart failure decompensation events and proximity to the patient, time of day, and level of severity of the predicted patient heart failure decompensation events. When implemented in a home setting, the intended recipient can be identified based on their capability to more quickly respond to any alerts that require immediate action.

In step 710, the alert and/or notification is transmitted to a device(s) associated with the predetermined recipient(s). Examples of devices associated with include their personal devices (with dedicated software installed) for receiving the alert and/or notification.

In step 712, remote application server 155 can receive and store the patient information, including the predicted patient heart failure decompensation events, the recommendations, and any other data associated with the patient, in a remote database, such as in the patient's EMR as described above in step 518.

### Sepsis Deployment

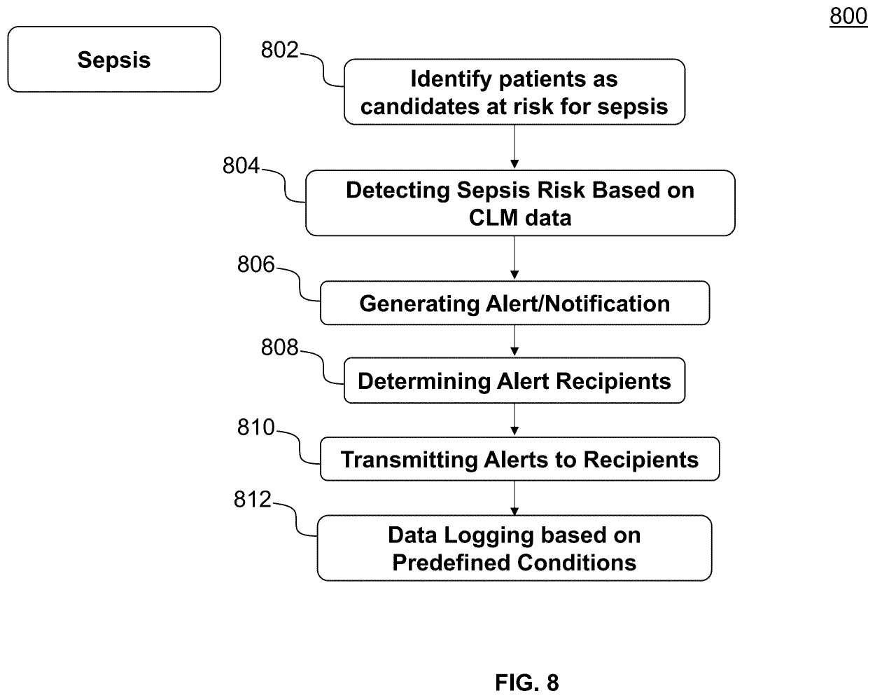

FIG. 8 is a flowchart illustrating a method 800 for utilizing CAM system 100 for the detection of sepsis conditions, according to some embodiments. Method 800 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 8 can be performed by a components of CAM system 100, either separately or in a distributed fashion. In such an embodiment, any of these components can execute code in memory to perform certain steps of method 800 of FIG. 8. While method 800 of FIG. 8 will be discussed below as being performed by certain components of CAM system 100, other components can store the code and therefore can execute method 800 by directly executing the code. Accordingly, the following discussion of method 800 will refer to components of FIGS. 1-3 as an exemplary non-limiting embodiment. Moreover, it is to be appreciated that not all steps can be needed to perform the disclosure provided herein. Further, some of the functions can be performed simultaneously, in a different order, or by the same components than shown in FIG. 8, as will be understood by a person of ordinary skill in the art.

Sepsis is the body's extreme response to an infection. It is a life-threatening medical emergency. Sepsis happens when an infection triggers an exaggerated reaction from the body's immune system, causing significant morbidity and mortality. Some people are at higher risk of developing sepsis because they are at higher risk of contracting an infection. These include the very young (infants), the very old, those with chronic illnesses, and those with a weakened or impaired immune system.

CAM system 100 is configured to measure lactate levels in dermal interstitial fluid allowing for closer monitoring of the lactate levels of patients suspected of having sepsis in the hospital and home settings. In particular, continuous lactate monitoring provides a novel means of measuring lactate levels for predicting the likelihood of sepsis in patients. CAM system 100 provides an increased level of surveillance of a patient's lactate levels with the potential of reducing the requirement for multiple blood tests, allowing for dynamic adjustments to treatment and therapy based on predicted likelihood of sepsis. A prediction model in CAM system 100 is configured to correlate rises in lactate levels with certain outcomes, including sepsis, and the prediction provided by the prediction model can be used to direct treatment. For example, during acute illnesses such as sepsis, levels of the hormone epinephrine are raised which leads to the conversion of glucose into lactate, increasing circulating the level of lactate in the patient. In addition, poor tissue oxygenation, primarily due to a drop in blood pressure, leads to anaerobic respiration which further contributes to lactate levels. The prediction model utilizes a patient's lactate information (e.g., columns 1006 from decision matrix 1000), including current levels and trending data along with data specific to the patient (e.g., trend data from column 1002 from decision matrix 1000) in order to generate a sepsis prediction.

Consequently, CAM system 100, when configured for continuous lactate measurement, can be used to predict the deterioration of a patient at risk of developing sepsis, monitor a patient's response to treatment of the sepsis, and provide a means of surveillance in patients recovering from sepsis who are at risk of recurrence, and predict additional clinical outcome based on the sepsis information and monitored information.

In step 802, a component of CAM system 100 (e.g., any combination of data receiving device 120, multi-purpose data receiving device 130, and/or user device 135) receives patient identification of any patients that are identified candidates for continuous remote monitoring based on a potential risk for sepsis. For example, during discharge from a hospital or during a visit to the doctor's visit, an HCP can determine that the patient is at risk for sepsis that would merit continuous remote monitoring including the continuous remote monitoring of a patient's analyte levels. Once identified, a patient identifier associated with the patient can be transmitted to CAM system 100 to indicate that the analyte sensor 102 associated with the patient is authorized to communicate with components of the CAM system 100 and should be used for predicting potential sepsis risk in the patient. In some embodiments, the prediction model relies on continuous lactate data (as opposed to a single measurement such as from a blood draw) which provides serial lactate measurements of a patient over a predetermined of time. Predictions of sepsis conditions can be more accurate when utilizing continuous lactate data.

In step 804, the prediction model can then utilize the CLM data for predicting potential sepsis conditions for the patient. The CLM data can be provided in the form of a cumulative sum of lactate levels over a predetermined period of time. The predetermined period of time be based on any number of factors include HCP preference, desired level of accuracy for the prediction, and determined severity of patient's condition. The prediction can also be based on prior treatment and the prediction model is providing a means of surveillance in patients recovering from sepsis and can be at risk of recurrence.

In step 806, a component of CAM system 100 (e.g., any combination of data receiving device 120, multi-purpose data receiving device 130, and/or user device 135) can generate an alert and/or notification based on the detection of a predicted sepsis condition. Alerts and notifications include the predicted sepsis condition. The alert can include one or more treatment recommendations for addressing the predicted sepsis condition. A component of CAM system 100 can generate the treatment recommendations based on the predicted patient outcome generated in 506 and include the generated recommendations in the alert. Accordingly, an alert and/or notification can be generated to notify one or more recipients of the patient's predicted sepsis condition.

In step 808, a component in CAM system 100 (e.g., any combination of data receiving device 120, multi-purpose data receiving device 130, and/or user device 135) can determine the recipients for receiving the generated alert and/or notification. The recipients can be predetermined based on different predicted sepsis condition, proximity to the patient, time of day, and level of severity of the predicted sepsis condition. When implemented in a home setting, the intended recipient can be identified based on their capability to more quickly respond to any alerts that require immediate action.

In step 810, the alert and/or notification is transmitted to a device associated with the predetermined recipient. Examples of devices associated with include their personal devices (with dedicated software installed) for receiving the alert and/or notification.

In step 812, remote application server 155 can receive and store the patient information including the predicted sepsis, the recommendations, and any other data associated with the patient, in a remote database, such as in the patient's EMR as described above in step 518.

### High Risk Surgery Deployment

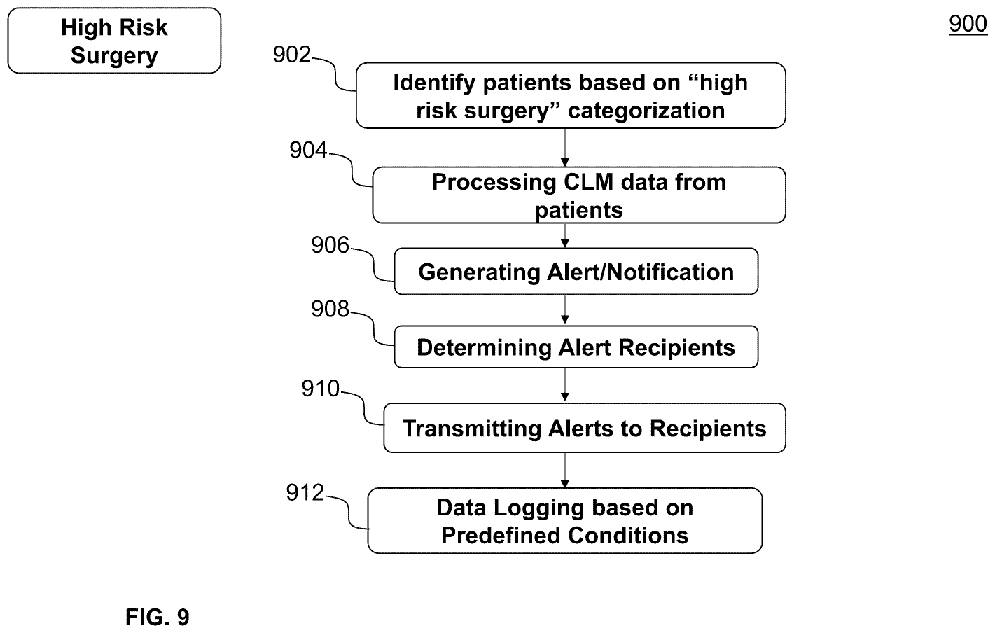

FIG. 9 is a flowchart illustrating a method 900 for utilizing a continuous analyte monitoring system during high-risk surgery procedures, according to some embodiments. Method 900 can be performed by processing logic that can comprise hardware (e.g., circuitry, dedicated logic, programmable logic, microcode, etc.), software (e.g., instructions executing on a processing device), or a combination thereof. As a non-limiting example of FIGS. 1-3, one or more functions described with respect to FIG. 9 can be performed by a components of CAM system 100, either separately or in a distributed fashion. In such an embodiment, any of these components can execute code in memory to perform certain steps of method 900 of FIG. 9. While method 900 of FIG. 9 will be discussed below as being performed by certain components of CAM system 100, other components can store the code and therefore can execute method 900 by directly executing the code. Accordingly, the following discussion of method 900 will refer to components of FIGS. 1-3 as an exemplary non-limiting embodiment. Moreover, it is to be appreciated that not all steps can be needed to perform the disclosure provided herein. Further, some of the functions can be performed simultaneously, in a different order, or by the same components than shown in FIG. 9, as will be understood by a person of ordinary skill in the art.

No surgery is without risk; however, some procedures have a higher risk of complication than others. Examples of surgical conditions which carry a high risk of developing perioperative complications, such as sepsis and hypovolemia, include, orthopaedic surgery, gastrointestinal surgery, oncologic surgery, vascular surgery, obstetric surgery, bariatric surgery, surgery in elderly patients, surgery in patients with comorbidities such as diabetes, heart failure, etc., and generally any surgery with prolonged anaesthesia.

Continuous lactate monitoring by CAM system 100 provides a means for real-time measurement of lactate levels in high-risk surgical settings. Rises in lactate levels are associated with worse clinical outcomes and continuous lactate monitoring could be used to detect patient decline earlier and to direct treatment decisions. A prediction model in CAM system 100 is configured to correlate rises in lactate levels with certain outcomes in high-risk surgical settings and the prediction provided by the prediction model can be used to anticipate potential patient conditions and dynamically adapt treatment of patients during their recovery period.

In step 902, CAM system 100 receives identification of any patients that have undergone surgical procedures categorized as “high-risk.” For example, before or after a high-risk surgical procedure, an HCP can identify the patient undergoing the procedure as a candidate for continuous monitoring of analyte levels by CAM system 100. Once identified, a patient identifier associated with the patient can be transmitted to CAM system 100 to indicate that the analyte sensor 102 associated with the patient is authorized to communicate with components, including the prediction model, of the CAM system 100.

In step 904, the prediction model can then utilize the CLM data for predicting potential deterioration conditions for the patient following the high-risk surgical procedure. The CLM data can be provided in the form of a cumulative sum of lactate levels over a predetermined period of time. The predetermined period of time be based on any number of factors include HCP preference, desired level of accuracy for the prediction, and determined severity of patient's condition. The prediction can also be based on the type of high-risk surgical procedure, any treatment occurring before and after the high-risk surgical procedure, and any other medical information associated with the patient including current vital signs. The prediction model is providing a means of surveillance in patients recovering from high-risk surgery and can be at risk of complications during recovery.

In step 906, a component of CAM system 100 (e.g., any combination of data receiving device 120, multi-purpose data receiving device 130, and/or user device 135) can generate an alert and/or notification based on the detection of any signs of deterioration in the patient. Alerts and notifications include the predicted deterioration conditions. The alert can include one or more treatment recommendations for addressing the predicted deterioration conditions. A component of CAM system 100 can generate the treatment recommendations based on the predicted deterioration conditions generated in 506 and include the generated recommendations in the alert. Accordingly, an alert and/or notification can be generated to notify one or more recipients of the patient's predicted deterioration conditions.

In step 908, a component in CAM system 100 (e.g., any combination of data receiving device 120, multi-purpose data receiving device 130, and/or user device 135) can determine the recipients for receiving the generated alert and/or notification. The recipients can be predetermined based on different predicted deterioration conditions, proximity to the patient, time of day, and level of severity of the predicted deterioration conditions. When implemented in a home setting (e.g., when the patient is recovering at home after the high-risk surgical procedure), the intended recipient can be identified based on their capability to more quickly respond to any alerts that require immediate action.

In step 910, the alert and/or notification is transmitted to a device associated with the predetermined recipient. Examples of devices associated with include their personal devices (with dedicated software installed) for receiving the alert and/or notification.

In step 912, remote application server 155 can receive and store the patient information, including the predicted patient deterioration conditions, the recommendations, and any other data associated with the patient, in a remote database, such as in the patient's EMR as described above in step 518.

The present disclosure describes various embodiments of a method for deploying CAM system 100 in a hospital setting (FIG. 5), a home setting (FIG. 6), for use with patients with potential heart conditions (FIG. 7), for use with patients with potential sepsis (FIG. 8), and for use with patients after certain medical procedures (FIG. 9). The steps described in each of these figures may be executed independently, sequentially, or in combination with steps described in other figures, based on detected conditions of the patient, HCP preferences, and/or configurable settings for the CAM system 100.

The combination of steps between each of these is based on the patient's medical information and medical history being provided by a common element CAM system 100 and that the patient's location (e.g., hospital, home) and medical conditions are continuously changing (e.g., between high-risk surgeries, potential sepsis, potential heart conditions).

For example, the method steps illustrated in FIG. 5 may be combined with one or more steps from FIGS. 6, 7, 9, and/or 9, such that [describe an example combination, e.g., patient monitoring during a high-risk surgery in FIG. 9 can trigger post-procedure monitoring for sepsis conditions with steps 806-810. These combinations may allow for an adaptive workflow that responds dynamically to patient vital signs, HCP observations, and patient procedures by automatically triggering steps from different deployments based on the current condition and location of the patient. Additionally, one or more steps from FIG. 5 or 6 may be interchanged, merged, omitted, or reordered with steps from FIG. 7, 8, or 9 while achieving a functionally equivalent result for each respective figure. For example, step 506 of FIG. 5 and step 606 of FIG. 6 (detecting signs of deterioration) may be embodied by steps of FIG. 7, 8, or 9, which results in identifying signs of deterioration with respect to heart failure, sepsis, or other conditions associated with high-risk surgery.

In another example, steps 802 and 804 of FIG. 8 (e.g., identifying candidates for risk of sepsis) may be performed automatically after certain high-risk procedures as described, for example, for FIG. 9. The same patient medical data (e.g., monitored lactate levels) and/or medical history (e.g., high-risk surgeries, other medical conditions) utilized with respect of FIG. 9 to notify an HCP that the patient is high risk (e.g., following steps 902-906) may be used for steps 804-810 of FIG. 8 after the patient has had the high-risk surgery by alerting the designated recipient that the patient is about to develop sepsis.

FIGS. 5-9 depict distinct implementations that can be modular and interoperable. Each element or process depicted in one figure may be used in conjunction with corresponding or alternative steps or sequence of steps from another figure if a patient meets criteria for different deployments. For example, a user may be admitted to the hospital (i.e., FIG. 5—hospital setting) while also diagnosed with heart conditions (i.e., FIG. 7), symptoms of sepsis (i.e., FIG. 8), and/or undergone certain high-risk procedures (i.e., FIG. 9). Similarly, a user may be discharged (i.e., FIG. 6—home setting) while also heart conditions, symptoms of sepsis, and/or undergone certain high-risk procedures.

In such embodiments, certain functionality is combinable such as generating and transmitting alerts and notifications to designated recipients. For example, detecting signs of deterioration in a hospital setting (i.e., step 506) where the deterioration involves a potential heart condition may trigger performance of steps 508-512 in addition to steps 706-710 for generating appropriate alerts and notifications to designated recipients for both the hospital setting deployment as well the heart failure deployment. As another example, step 702 for collecting continuous lactate monitoring data may result in triggering different steps for predicting both heart failure conditions of FIG. 7 as well as identifying potential sepsis symptoms of FIG. 8 based on the same collected data.

Further, the steps shown in any one figure may be selectively activated, omitted, modified, or combined to accommodate different patient conditions, medical history, specific patient requirements, and alternative hardware implementations. In some embodiments, the described method may be implemented using a rules-based engine, machine learning model, or predefined logic to dynamically determine whether and how steps from different figures should be executed together.

Unless otherwise explicitly stated, the methods and components depicted in FIGS. 5-9 should be understood as interoperable, interchangeable, and combinable, meaning that one or more elements of one figure may be integrated with or adapted to work with elements from another figure without deviating from the scope of the invention. The specific combinations described herein are provided for illustrative purposes and are not intended to be exhaustive or limiting.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

Exemplary embodiments are set forth in the following numbered clauses:
1. A method for monitoring a patient based on continuous analyte information, the method comprising:
receiving the continuous analyte information, wherein the continuous analyte information is associated with a currently admitted patient, and wherein the continuous analyte information is provided by a continuous analyte sensor associated with the currently admitted patient;
monitoring the continuous analyte information for analyte level and rate of change of analyte level of the currently admitted patient;
correlating analyte level and rate of change of analyte level of the currently admitted patient;
generating a predicted patient outcome based on the correlation; and
transmitting a notification to a recipient, wherein the notification includes the predicted patient outcome and a recommendation on whether to discharge the patient from the hospital.
2. The method of clause 1, wherein the analyte comprises lactate.
3. The method of clause 1 or 2, wherein the correlation of analyte level and rate of change of analyte level of the currently admitted patient is performed by matching a first patient identifier associated with the analyte level to a second patient identifier associated with the rate of change of analyte level.
4. The method of clause 3, wherein the generation of a predicted patient outcome based on the correlation is done by:
comparing the matched patient identifiers with a decision matrix wherein each point on the decision matrix is associated with a predicted patient outcome;
determining the predicted patient outcome associated with the matched patient identifiers based on the decision matrix; and dynamically updating the decision matrix based on additional patient data.
5. The method of any one of clauses 1 to 4, wherein the recommendation is based on patient medical history comprising an indication that the currently admitted patient had suffered from a condition within a predetermined period of time.
6. The method of clause 5, wherein the condition comprises sepsis or septic shock; infection; hypoxia; heart failure; polytrauma; tissue hypoperfusion; pulmonary, circulatory, neurological, hepatic, and/or renal systems disorders; and liver or/and kidney diseases.
7. The method of any one of clauses 1 to 6, wherein the predicted patient outcome is further based on additional factors, and wherein the method further comprises: inputting, to a patient prediction model, the additional factors in combination with the continuous analyte information, wherein the additional factors include one or more of: patient medical history, physical exertion/exercise, patient procedure history, current vital signs, genomics, comorbidities, body temperature, pulse rate, breathing rate, blood pressure, biometrics, age, trend information, and historical data and trends of previous patients.
8. The method of clause 7, wherein one or more factors of the additional factors are weighted based on a current condition of the currently admitted patient.
9. The method of any one of clauses 1 to 7, wherein the first graphical visualization further comprises a visualization of a trend information of the analyte level information over a period of time.
10. The method any one of clauses 1 to 8, wherein the second graphical visualization comprises the visual representation of the trend information of the analyte level information and the monitoring device is a bedside monitor associated with the currently admitted patient.
11. The method of clause 10, further comprising:
receiving, by the monitoring device, a display request from a user device associated with a healthcare provider of the currently admitted patient, wherein display request includes a selection of one of the analyte level information and the rate of change information; and displaying the one of the analyte level information and the rate of change information on the monitoring device based on the display request, wherein the one of the analyte level information and the rate of change information is displayed until receipt of a cancel display request.
12. The method of any one of clauses 1 to 9, wherein the recommendation is to discharge the currently admitted patient from the hospital.
13. The method of clause 12, wherein the recommendation further comprises additional instructions to be performed by the currently admitted patient after discharge, wherein the additional instructions comprises a second recommendation for wearing a continuous analyte sensor for a predetermined period of time after discharge
14. The method of any of clauses 1 to 10, wherein the recommendation is to prevent discharging the currently admitted patient at the hospital for continued treatment and/or observation.
15. A system for monitoring a patient based on continuous analyte information, the system is configured to:
receive the continuous analyte information, wherein the continuous analyte information is associated with a currently admitted patient, and wherein the continuous analyte information is provided by a continuous analyte sensor associated with the currently admitted patient;
process the continuous analyte information for analyte level information and rate of change information of the currently admitted patient;
generate a predicted patient outcome based on the analyte level information and the rate of change information;
generate a first graphical visualization and a second graphical visualization based on the predicted patient outcome and at least one of a current patient condition of the currently admitted patient, historical patient condition of the currently admitted patient, and a current disease state of the currently admitted patient;
transmit a first notification comprising the first graphical visualization to a recipient device, wherein the first graphical visualization includes the predicted patient outcome and a recommendation on whether to discharge the patient; and
modify a graphical user interface of a monitoring device to display the second graphical visualization.
16. The system of clause 15, wherein the analyte comprises lactate.
17. The system of clause 15, wherein the correlation of analyte level and rate of change of analyte level of the currently admitted patient is performed by matching a patient identifier associated with the analyte level to a patient identifier associated with the rate of change of analyte level.
18. The system of clause 15, wherein the generation of a predicted patient outcome based on the correlation is done by:
comparing the matched patient identifiers with a decision matrix wherein each point on the decision matrix is associated with a predicted patient outcome; and
determining the predicted patient outcome associated with the matched patient identifiers based on the decision matrix.
19. The system of any one of clauses 15-18, wherein the recommendation is based on patient medical history comprising an indication that the currently admitted patient had suffered from a condition within a predetermined period of time.
20. The system of clause 19, wherein the condition comprises sepsis or septic shock; infection; hypoxia; heart failure; polytrauma; tissue hypoperfusion; pulmonary, circulatory, neurological, hepatic, and/or renal systems disorders; and liver or/and kidney diseases.
21. The system of any one of clauses 15 to 20, wherein the predicted patient outcome is further based on additional factors, and wherein the system is further configured to: input, to a patient prediction model, the additional factors in combination with the continuous analyte information, wherein the additional factors include one or more of: patient medical history, physical exertion/exercise, patient procedure history, current vital signs, genomics, comorbidities, body temperature, pulse rate, breathing rate, blood pressure, biometrics, age, trend information, and historical data and trends of previous patients
22. The system of any one of clauses 15 to 21, wherein the notification is selected from one or more of the group comprising a text message, a voice memo, a number value, and a visual display such as a trend graph or a table.
23. The system of any one of clauses 15 to 22, wherein the notification may additionally comprise one or more of an alert indicating recommended actions, information about current and/or predicted conditions of the currently admitted patient, current and/or predicted trend graphs, current and/or predicted analyte levels, and visual information.
24. The system of any one of clauses 15 to 23, wherein the recommendation is to discharge the currently admitted patient from the hospital.
25. A method for monitoring a patient based on continuous analyte information, the method comprising:
receiving the continuous analyte information, wherein the continuous analyte information is associated with a patient who had been discharged from a hospital within the past 30 days, and wherein the continuous analyte information is provided by a continuous analyte sensor associated with the currently admitted patient;
monitoring the continuous analyte information for analyte level and rate of change of analyte level of the patient;
correlating analyte level and rate of change of analyte level of the patient;
generating a predicted patient outcome based on the correlation; and
transmitting a notification to a recipient, wherein the notification includes the predicted patient outcome and a recommendation on a course of action not necessitating readmission to the hospital or to readmit the patient to the hospital.
26. The method of clause 25, wherein the analyte comprises lactate.
27. The method of clause 25, wherein the correlation of analyte level and rate of change of analyte level of the currently admitted patient is performed by matching a patient identifier associated with the analyte level to a patient identifier associated with the rate of change of analyte level.
28. The method of clause 27, wherein the generation of a predicted patient outcome based on the correlation is done by:
comparing the matched patient identifiers with a decision matrix wherein each point on the decision matrix is associated with a predicted patient outcome; and
determining the predicted patient outcome associated with the matched patient identifiers based on the decision matrix.
29. The method of any one of clauses 25 to 28, wherein the recommendation is based on patient medical history comprising an indication that the patient had suffered from a condition within a predetermined period of time.
30. The method of clause 29, wherein the condition comprises sepsis or septic shock; infection; hypoxia; heart failure; polytrauma; tissue hypoperfusion; pulmonary, circulatory, neurological, hepatic, and/or renal systems disorders; and liver or/and kidney diseases.
31. The method of any one of clauses 25 to 30, wherein the predicted patient outcome is further based on additional factors, and wherein the method further comprises: inputting, to a patient prediction model, additional factors in combination with the continuous analyte information, wherein the additional factors include one or more of: patient medical history, physical exertion/exercise, patient procedure history, current vital signs, genomics, comorbidities, body temperature, pulse rate, breathing rate, blood pressure, biometrics, age, trend information, and historical data and trends of previous patients.
32. The method of any one of clauses 25 to 31, wherein the notification is selected from one or more of the group comprising a text message, a voice memo, a number value, and a visual display such as a trend graph or a table.
33. The method of any one of clauses 25 to 32, wherein the notification may additionally comprise one or more of an alert indicating recommended actions, information about current and/or predicted conditions of the patient, current and/or predicted trend graphs, current and/or predicted analyte levels, and visual information.
34. The method of any one of clauses 25 to 33, wherein the recommendation comprises an instruction to readmit the patient to the hospital.
35. The method of any one of clauses 25 to 33, wherein the recommendation comprises a course of action not necessitating readmission to the hospital.
36. A method for monitoring a patient based on continuous analyte information, the method comprising:
receiving the continuous analyte information, wherein the continuous analyte information is associated with a patient, and wherein the continuous analyte information is provided by a continuous analyte sensor associated with the patient;
monitoring the continuous analyte information for analyte level and rate of change of analyte level of the patient;
correlating analyte level and rate of change of analyte level of the patient;
generating a predicted patient outcome based on the correlation; and
transmitting a notification to a recipient, wherein the notification includes the predicted patient outcome.
37. The method of clause 36, wherein the analyte comprises lactate.
38. A system for monitoring a patient based on continuous analyte information, the system is configured to:
receive the continuous analyte information, wherein the continuous analyte information is associated with a patient, and wherein the continuous analyte information is provided by a continuous analyte sensor associated with the patient;
monitor the continuous analyte information for analyte level and rate of change of analyte level of the patient;
correlate analyte level and rate of change of analyte level of the patient;
generate a predicted patient outcome based on the correlation; and
transmitting a notification to a recipient, wherein the notification includes the predicted patient outcome.
39. The system of clause 38, wherein the analyte comprises lactate.

Additional exemplary embodiments are set forth in the following numbered clauses:
40. A computer implemented method for predicting patient outcome based on continuous analyte data, the method comprising:
receiving, by a processor implemented in an early warning system, the continuous analyte data, wherein the continuous analyte data is associated with a patient, and wherein the continuous analyte data is provided by a continuous analyte sensor associated with the patient;
generating, by the processor, a predicted patient outcome based on the continuous analyte data;
identifying, by the processor and based on the predicted patient outcome and the continuous analyte data, a predetermined recipient device; and
transmitting, by the processor, a notification to the predetermined recipient device, wherein the notification includes the predicted patient outcome.
41. The method of 40, wherein the processor is in communication with a patient prediction model, and wherein the patient prediction model is a machine learning model.
42. The method of 41, wherein the predicted patient outcome is generated by the patient prediction model, wherein the method further comprises:
inputting, to the patient prediction model, the continuous analyte data; and
outputting, by the patient prediction model to the processor, the predicted patient outcome.
43. The method of any preceding clause, wherein the notification further includes a recommendation for treating the predicted patient outcome.
44. The method of any preceding clause, wherein the predicted patient outcome is further based on patient medical information, and wherein the method further comprises: inputting, to the patient prediction model, the patient medical information in combination with the continuous analyte data, wherein the patient medical information includes one or more of patient procedure history, patient medical history, or current vital signs associated with the patient.
45. The method of any preceding clause, further comprising retrieving, by the processor, a use-case deployment associated with the patient, and wherein the use-case deployment includes one of a hospital setting, a home setting, a sepsis condition, a heart failure condition, or a high-risk surgery condition.
46. The method of any preceding clause, wherein the analyte sensor is a dual-analyte sensor for detecting a first and second continuous analyte data.
47. The method of 46, wherein the first and second continuous analyte data comprise any combination of two analytes from lactate, glucose, creatinine, and ketone.
48. The method of v, further comprising determining a trend in a value of the analyte and the analyte based on the continuous analyte data, and generating the predicted patient outcome based on both the determined trend and the analyte value.
49. The method of any preceding clause, further comprising determining a trend in a value of the analyte and the analyte value based on the continuous analyte data, and determining the content of the notification and/or recipient of the notification based on both the determined trend and the analyte value.
50. The method of 48 or 49, wherein:
when the determined analyte value is below a first threshold and the determined trend is above a second threshold, the method comprises sending an alert to a device associated with a determined recipient; and
when the determined analyte value is below the first threshold and the determined trend is below the second threshold, no alert is sent.
51. The method of 50, wherein:
when the determined analyte value is below the first threshold, the determined trend is above the second threshold, and the use-case deployment is a home setting, the method comprises sending an alert to a device associated with a determined recipient;
when the determined analyte value is below the first threshold, the determined trend is above the second threshold but below a third threshold higher than the second threshold, and the use-case deployment is a hospital setting, no alert is sent; and when the determined analyte value is below the first threshold, the determined trend is above the third threshold, and the use-case deployment is a hospital setting, the method comprises sending an alert to a device associated with a determined recipient.
52. The method of 50 or 51, wherein:
when the determined analyte value is above a fourth threshold and the determined trend is above a fifth threshold, the method comprises sending an alert to a device associated with a determined recipient; and
when the determined analyte value is above the fourth threshold and the determined trend is below the fifth threshold, no alert is sent.
53. The method of any preceding clause, wherein the trend is a rate of change of the value of the analyte over a given time period, wherein the given time period comprises fifteen minutes, one hour, two hours, or three or more hours.
54. The method of any preceding clause, further comprising determining the content of the notification and/or recipient of the notification based on the predicted patient outcome and/or determined use-case deployment.
55. The method of 54, wherein the content of the notification and/or recipient of the notification is determined based on one or more of the proximity of the recipient to the patient, time of day, and level of severity of the predicted patient outcome.
56. The method of 54 or 55, wherein the notification includes an instruction to administer an intervention to the patient based on the predicted patient outcome, and the recipient of the notification is a medical device configured to automatically administer the intervention in response to the instruction.
57. The method of 536 wherein the intervention is one or more of fluid resuscitation, administration of antibiotics, or provision of breathing oxygen gas.
58. The method of any preceding clause, wherein the notification includes an instruction for adjusting or maintaining a dosage of a substance to be administered to the patient, and the recipient of the notification is a device configured to administer the substance to the patient based on the dosage specified in the instruction.
59. The method of 58, wherein the treatment device is an insulin injection pen or insulin pump and the analyte is glucose.
60. The method of any preceding clause, wherein notifications are sent at a frequency based on the use-case deployment.
61. The method of any preceding clause, wherein the continuous analyte data is received at a frequency based on the determined trend, the frequency configured to increase when the determined trend is above a another threshold.
62. The method of any preceding clause, wherein the notification comprises an alert notifying a caregiver and/or the patient that an immediate intervention is required.
63. The method of any preceding clause, wherein the predicted patient outcome comprises a determination of a particular disease, optionally heart failure or sepsis.
64. A computer-implemented method for operating an early warning system based on continuous analyte data, the method comprising:
receiving, by a processor implemented in the early warning system, first continuous analyte data and second continuous analyte data, wherein the first continuous analyte data is associated with a first patient and the second continuous analyte data is associated with a second patient, and wherein the first continuous analyte data is provided by a first continuous analyte sensor associated with the first patient and the second continuous analyte data is provided by a second continuous analyte sensor associated with the second patient;
determining, by the processor in communication with the first continuous analyte sensor and the second continuous analyte sensor, a first use-case deployment based on at least one of a first user preference and a monitored condition of the first patient and a second use-case deployment based on at least one a second user preference and a monitored condition of the second patient;
providing, by the processor, the first use-case deployment and the first continuous analyte data as a first input to a patient prediction model;
providing, by the processor, the second use-case deployment and the second continuous analyte data as a second input to the patient prediction model, wherein the first use-case deployment differs from the second use-case deployment;
receiving, from the patient prediction model, a first predicted patient outcome associated with the first patient and a second predicted patient outcome associated with the second patient;
identifying, based on the first predicted patient outcome, a first predetermined recipient device, and based on the second predicted patient, a second predetermined recipient device; and
transmitting a first notification, generated based on the first predicted patient outcome, to the first predetermined remote device, and a second notification, generated based on the second predicted patient outcome, to the second predetermined remote device.
65. The method of 64, wherein the patient prediction model is a machine learning model.
66. The method of 64 or 65, wherein the notification further includes a recommendation for treating the predicted patient outcome.
67. The method of any preceding clause, wherein the predicted patient outcome is generated by a patient prediction model, wherein the method further comprises: inputting, to the patient prediction model, the continuous analyte data; and outputting, by the patient prediction model, the predicted patient outcome.
68. The method of any preceding clause, wherein the predicted patient outcome is further based on patient medical information, and wherein the method further comprises:
inputting, to the patient prediction model, the patient medical information in combination with the continuous analyte data, wherein the patient medical information includes one or more of patient procedure history, patient medical history, or current vital signs associated with the patient.
69. The method of any preceding clause, wherein the use-case deployment includes one of a hospital setting, a home setting, a sepsis condition, a heart failure condition, or a high-risk surgery condition.
70. The method of any preceding clause, wherein the analyte sensor is a dual-analyte sensor and the first and second continuous analyte data comprise any combination of two analytes from lactate, glucose, creatinine, and ketone.
71. The method of any preceding clause, further comprising determining a trend in a value of the analyte and an analyte value based on the continuous analyte data, and generating the predicted patient outcome based on both the determined trend and analyte value.
72. The method of any preceding clause, further comprising determining a trend in a value of the analyte and an analyte value based on the continuous analyte data, and determining the content of the notification and/or recipient of the notification based on both the determined trend and analyte value.
73. The method of any preceding clause, wherein:
when the determined analyte value is below a first threshold and the determined trend is above a second threshold, the method comprises sending an alert to a device associated with a determined recipient; and
when the determined analyte value is below the first threshold and the determined trend is below the second threshold, no notification is transmitted.
74. The method of 71, wherein:
when the determined analyte value is below the first threshold, the determined trend is above the second threshold, and the use-case deployment is a home setting, the method comprises sending an alert to a device associated with a determined recipient;
when the determined analyte value is below the first threshold, the determined trend is above the second threshold but below a third threshold higher than the second threshold, and the use-case deployment is a hospital setting, no alert is sent; and
when the determined analyte value is below the first threshold, the determined trend is above the third threshold, and the use-case deployment is a hospital setting, the method comprises sending an alert to a device associated with a determined recipient.
75. The method of any preceding clause, wherein:
when the determined analyte value is above a fourth threshold and the determined trend is above a fifth threshold, the method comprises sending an alert to a device associated with a determined recipient; and
when the determined analyte value is above the fourth threshold and the determined trend is below the fifth threshold, no alert is sent.
76. The method of any preceding clause, wherein the trend is a rate of change of the value of the analyte over a given time period, wherein the given time period comprises fifteen minutes, one hour, two hours, or three or more hours.
77. The method of any preceding clause, further comprising determining the content of the notification and/or recipient of the notification based on the predicted patient outcome and/or determined use-case deployment.
78. The method of 77, wherein the content of the notification and/or recipient of the notification is determined based on one or more of the proximity of the recipient to the patient, time of day, and level of severity of the predicted patient outcome.
79. The method of any preceding clause, wherein the notification includes an instruction to administer an intervention to the patient based on the predicted patient outcome, and the recipient of the notification is a medical device connected to the patient and configured to automatically administer the intervention in response to the instruction.
80. The method of 79, wherein the intervention is one or more of fluid resuscitation, administration of antibiotics, or provision of breathing oxygen gas.
81. The method of any preceding clause, wherein the notification includes an instruction for adjusting or maintaining a dosage of a substance to be administered to the patient, and the recipient of the notification is a device configured to administer the substance to the patient based at the dosage specified in the instruction.
82. The method of 81, wherein the treatment device is an insulin injection pen or insulin pump and the analyte is glucose.
83. The method of any preceding clause, wherein notifications are sent at a frequency based on the use-case deployment.
84. The method of any preceding clause, wherein the continuous analyte data is received at a frequency based on the determined trend, the frequency configured to increase when the determined trend is above a given threshold.
85. The method of any preceding clause, wherein the notification comprises an alert notifying a caregiver and/or the patient that an immediate intervention is required.
86. An early warning system configured to perform the method of any preceding clause.
87. An early warning system configured to perform the method of any preceding clause, wherein the prediction model is implemented on any of one combination of a data receiving device, multi-purpose data receiving device, and/or user device.
88. An early warning system configured to perform the method of any preceding clause, wherein the prediction model comprises at least one of first prediction model configured for a hospital deployment, a second prediction model configured for a home deployment, a third prediction model configured for a heart failure deployment, a fourth prediction model configured for a sepsis deployment, and a fifth prediction model configured for a high-risk surgery deployment.

What is claimed is:

1. A method for monitoring a patient based on continuous analyte information, the method comprising:
receiving the continuous analyte information, wherein the continuous analyte information is associated with a currently admitted patient, and wherein the continuous analyte information is provided by a continuous analyte sensor associated with the currently admitted patient;
processing the continuous analyte information for analyte level information and rate of change information of the currently admitted patient;
generating a predicted patient outcome based on the analyte level information and the rate of change information;
generating a first graphical visualization and a second graphical visualization based on the predicted patient outcome and at least one of a current patient condition of the currently admitted patient, historical patient condition of the currently admitted patient, and a current disease state of the currently admitted patient;
transmitting a first notification comprising the first graphical visualization to a recipient device, wherein the first graphical visualization includes the predicted patient outcome and a recommendation on whether to discharge the currently admitted patient; and
modifying a graphical user interface of a monitoring device to display the second graphical visualization.

2. The method of claim 1, wherein the continuous analyte information comprises lactate information.

3. The method of claim 1, wherein the generating the predicted patient outcome is based on matching a first patient identifier associated with the analyte level information to a second patient identifier associated with the rate of change information.

4. The method of claim 3, wherein generating the predicted patient outcome further comprises:
comparing the matched patient identifier with decision matrix wherein each point on the decision matrix is associated with the predicted patient outcome;
determining the predicted patient outcome associated with the matched patient identifiers based on the decision matrix; and
dynamically updating the decision matrix based on additional patient data.

5. The method of any one of claim 1, wherein the recommendation is based on patient medical history comprising an indication that the currently admitted patient had suffered from a condition within a predetermined period of time.

6. The method of claim 5, wherein the condition comprises sepsis or septic shock; infection; hypoxia; heart failure; polytrauma; tissue hypoperfusion; pulmonary, circulatory, neurological, hepatic, and/or renal systems disorders; and liver or/and kidney diseases.

7. The method of claim 1, wherein the predicted patient outcome is further based on additional factors, and wherein the method further comprises:
inputting, to a patient prediction model, the additional factors in combination with the continuous analyte information, wherein the additional factors include one or more of: the patient medical history, physical exertion/exercise, patient procedure history, current vital signs, genomics, comorbidities, body temperature, pulse rate, breathing rate, blood pressure, biometrics, age, trend information, and historical data and trends of previous patients.

8. The method of claim 7, wherein one or more factors of the additional factors are weighted based on a current condition of the currently admitted patient.

9. The method of claim 1, wherein the first graphical visualization further comprises a visualization of a trend information of the analyte level information over a period of time.

10. The method of claim 1, wherein the second graphical visualization comprises the visual representation of the trend information of the analyte level information and the monitoring device is a bedside monitor associated with the currently admitted patient.

11. The method of claim 10, further comprising:
receiving, by the monitoring device, a display request from a user device associated with a healthcare provider of the currently admitted patient, wherein display request includes a selection of one of the analyte level information and the rate of change information; and
displaying the one of the analyte level information and the rate of change information on the monitoring device based on the display request, wherein the one of the analyte level information and the rate of change information is displayed until receipt of a cancel display request.

12. The method of claim 1, wherein the recommendation is to discharge the currently admitted patient.

13. The method of claim 12, wherein the recommendation further comprises additional instructions to be performed by the currently admitted patient after discharge, wherein the additional instructions comprises a second recommendation for wearing a continuous analyte sensor for a predetermined period of time after discharge.

14. The method of claim 1, wherein the recommendation is to prevent discharging the currently admitted patient for continued treatment and/or observation.

15. A system for monitoring a patient based on continuous analyte information, the system is configured to:
receive the continuous analyte information, wherein the continuous analyte information is associated with a currently admitted patient, and wherein the continuous analyte information is provided by a continuous analyte sensor associated with the currently admitted patient;
process the continuous analyte information for analyte level information and rate of change information of the currently admitted patient;
generate a predicted patient outcome based on the analyte level information and the rate of change information;
generate a first graphical visualization and a second graphical visualization based on the predicted patient outcome and at least one of a current patient condition of the currently admitted patient, historical patient condition of the currently admitted patient, and a current disease state of the currently admitted patient;
transmit a first notification comprising the first graphical visualization to a recipient device, wherein the first graphical visualization includes the predicted patient outcome and a recommendation on whether to discharge the patient; and
modify a graphical user interface of a monitoring device to display the second graphical visualization, wherein the graphical user interface further comprises an interactive graphical visualization configured to receive additional user input associated with the currently admitted patient.

16. The system of claim 15, wherein the continuous analyte information comprises lactate information.

17. The system of claim 15, wherein the predicted patient outcome is further based on additional factors, and wherein the system is further configured to:
input, to a patient prediction model, the additional factors in combination with the continuous analyte information, wherein the additional factors include one or more of: patient medical history, physical exertion/exercise, patient procedure history, current vital signs, genomics, comorbidities, body temperature, pulse rate, breathing rate, blood pressure, biometrics, age, trend information, and historical data and trends of previous patients

18. The system of claim 15, wherein the recommendation is to discharge the currently admitted patient.

19. A method for monitoring a patient based on continuous analyte information, the method comprising:
receiving the continuous analyte information, wherein the continuous analyte information is associated with a currently admitted patient who had been discharged from a hospital within a predetermined period of time, and wherein the continuous analyte information is provided by a continuous analyte sensor associated with the currently admitted patient;
processing the continuous analyte information of the currently admitted patient for analyte level information and rate of change information of the currently admitted patient;
generating a predicted patient outcome based on the analyte level information and the rate of change information;
generating a first graphical visualization and a second graphical visualization based on the predicted patient outcome and at least one of a current patient condition of the currently admitted patient, historical patient condition of the currently admitted patient, and a current disease state of the currently admitted patient;
transmitting a first notification comprising the first graphical visualization to a recipient device, wherein the first graphical visualization includes the predicted patient outcome and a recommendation on whether to discharge the currently admitted patient; and
modifying a graphical user interface of a monitoring device to display the second graphical visualization.

20. The method of claim 19, wherein the continuous analyte information comprises lactate information.

21. The method of claim 19, wherein the predicted patient outcome is further based on additional factors, and wherein the method further comprises:
inputting, to a patient prediction model, the additional factors in combination with the continuous analyte information, wherein the additional factors include one or more of: patient medical history, physical exertion/exercise, patient procedure history, current vital signs, genomics, comorbidities, body temperature, pulse rate, breathing rate, blood pressure, biometrics, age, trend information, and historical data and trends of previous patients.

22. The method of claim 19, wherein the recommendation comprises an instruction to readmit the currently admitted patient to the hospital.

23. The method of claim 19, wherein the recommendation comprises a course of action not necessitating readmission to the hospital.

24. An early warning system, comprising:
a continuous analyte sensor configured to continuously collect continuous analyte data of a patient;
one or more processors in communication with the continuous analyte sensor; and
a memory coupled to the one or more processors and storing a prediction model and instructions that when executed by the one or more processors cause the one or more processors to:
receive the continuous analyte data;
determine a use-case deployment based on at least one of a user preference and a monitored condition of the patient;
determine a trend in the continuous analyte data and an analyte value based on the continuous analyte data;
provide the use-case deployment, the determined trend, the analyte value, and the continuous analyte data as inputs to a patient prediction model;
receive, from the patient prediction model, a predicted patient outcome associated with the patient;
identify a predetermined recipient device based on the predicted patient outcome; and
transmit a notification, generated based on the predicted patient outcome, to the predetermined recipient device.

25. A method for operating an early warning system based on continuous analyte data, the method comprising:
receiving, by a processor implemented in the early warning system, continuous analyte data, wherein the continuous analyte data is associated with a patient, and wherein the continuous analyte data is provided by a continuous analyte sensor associated with the patient;
determining, by the processor in communication with the continuous analyte sensor, a use-case deployment based on at least one of a user preference and a monitored condition of the patient;
determining a trend in the continuous analyte data and an analyte value based on the continuous analyte data;
providing, by the processor, the use-case deployment, the determined trend, the analyte value, and the continuous analyte data as inputs to a patient prediction model;
receiving, from the patient prediction model, a predicted patient outcome associated with the patient;
identifying a predetermined recipient device based on the predicted patient outcome; and
transmitting a notification, generated based on the predicted patient outcome, to the predetermined recipient device.

26. The method of claim 25, wherein the notification comprises a recommendation for treating the predicted patient outcome.

27. The method of claim 26, wherein the predicted patient outcome is generated by the patient prediction model, wherein the method further comprises:
inputting, to the patient prediction model, the continuous analyte data; and
outputting, by the patient prediction model, the predicted patient outcome.

28. The method of claim 26, wherein the predicted patient outcome is further based on patient medical information, and wherein the method further comprises:
inputting, to the patient prediction model, patient medical information in combination with the continuous analyte data, wherein the patient medical information includes one or more of patient procedure history, patient medical history, or current vital signs associated with the first patient.

29. The method of claim 25, wherein the notification includes an instruction for adjusting or maintaining a dosage of a substance to be administered to the patient, and the predetermined recipient device is configured to administer the substance to the patient based at the dosage specified in the instruction.

30. The method of claim 25, wherein content of the notification is determined based on one or more of a proximity of a recipient of the predetermined recipient device to the patient, a time of day, and level of severity of the predicted patient outcome and identifying the predetermined recipient device is further based on one or more of the proximity of the recipient to the patient, the time of day, and the level of severity of the predicted patient outcome.

31. The method of claim 25, wherein:
when the analyte value in the continuous analyte data is below a first threshold and the determined trend is above a second threshold, the method comprises sending, by the processor, an alert to the predetermined recipient device; and
when the analyte value is below the first threshold and the determined trend is below the second threshold, preventing transmission of the alert.

32. The method of claim 31, wherein:
when the analyte value is below the first threshold, the determined trend is above the second threshold, and the use-case deployment is a home setting, the method comprises sending a second alert to a device associated with another predetermined recipient device;
when the analyte value is below the first threshold, the determined trend is above the second threshold but below a third threshold higher than the second threshold, and the use-case deployment is a hospital setting, preventing transmission of the second alert; and
when the analyte value is below the first threshold, the determined trend is above the third threshold, and the use-case deployment is the hospital setting, the method comprises sending an alert to a device associated with a different predetermined recipient device.

33. The method of claim 25, wherein the determined trend is a rate of change of the analyte value over a given time period.

34. The method of claim 25, further comprising determining content of the notification based on the predicted patient outcome and the use-case deployment.

35. The method of claim 25, wherein a content of the notification is determined based on one or more of a proximity of a recipient associated with the predetermined recipient device to the patient, time of day, and a level of severity of the predicted patient outcome.

36. The method of claim 3, wherein the content of the notification includes an instruction to administer an intervention to the patient based on the predicted patient outcome, and the predetermined recipient device is configured to automatically administer the intervention in response to the instruction.
